(19) United States
(12) Patent Application Publication
(10) Pub. No.: US 2005/0010118 A1
(43) Pub. Date: Jan. 13, 2005

(54) Method and device for measuring pulse rate, blood pressure, and monitoring blood vessel access

(75) Inventors: Masahiro Toyoda, Haibara-gun (JP); Hiromi Yamazaki, Haibara-gun (JP)

Correspondence Address: DARBY & DARBY P.C., P. O. BOX 5257, NEW YORK, NY, 10150-5257, US

(73) Assignee: Nikkiso Co. Ltd., Tokyo (JP)

(21) Appl. No.: 10/887,752

(22) Filed: Jul. 9, 2004

(30) Foreign Application Priority Data
Jul. 10, 2003 (JP) 2003-194932
Jul. 10, 2003 (JP) 2003-194931

(51) Int. Cl. A61B 5/02

(52) U.S. Cl. 600/486

(57) ABSTRACT

A method and device for measuring a patient's pulse rate and blood pressure and also the condition of the blood vessel access can be accurately monitored by identifying a frequency component of the pressure wave caused by the patient's heartbeat among other pressure waves in a fluid by frequency analysis. The method and device are used when a medical device is connected to the patient's blood vessel via a blood vessel access and has a mechanical device for applying pressure to a fluid to transport it to said blood vessel.

The present application claims priority under 35 U.S.C. §119 to Japanese Patent Application Nos. 2003-194931 filed on Jul. 10, 2003 and 2003-194932 filed on Jul. 10, 2003. The contents of the applications are incorporated herein by reference in their entireties.

### TECHNICAL FIELD

The invention relates to a method and device for measuring pulse rate, blood pressure, and monitoring blood vessel access in reference to medical treatments using various devices such as dialysis devices, artificial heart-lung machines, and infusion pumps.

### BACKGROUND ART

Dialysis devices, artificial heart-lung machines, or infusion pumps have been known as medical devices for transporting to or circulating through patients' blood vessels via blood vessel access. In case of a dialysis device, for example, it is connected to the patient's blood vessel via a blood vessel access and the fluid that is transported to blood vessels via a blood vessel access is blood, thus constituting a system to return the blood back to the body after removing wastes accumulated in the blood by device of the dialysis device and circulating the blood. Also, in case of a infusion pump, it is connected to the patient's blood vessel via a blood vessel access and a fluid medicine or a nutrient fluid is transported to the blood vessel and thus the fluid medicine or the nutrient fluid is infused into the patient's body.

In case of a patient connected to a dialysis device under a dialysis treatment, waste substances and water in the blood are removed an osmotic pressure difference between the dialysate flowing outside of a hollow fiber provided in the dialyzer and the blood flowing inside of the hollow fiber as well as ultrafiltration as the blood is taken out of the patient's body and pushed through the dialyzer by the operation of the pump. During an dialysis therapy process, the pulse rate and blood pressure of the patient can sometimes change very abruptly. Therefore, it is customary to have nurses or other medical professionals to check the conditions and measure pulse rates and blood pressures of the patients under extracorporeal dialysis regularly, typically once an hour. The measurement of pulse rates is typically done by a nurse or other medical professionals by placing a finger on one of the arteries of the patient's arm, detecting the pulse by tactile sensing, and counting the number of pulses within a unit time while measuring the time by a wrist watch, etc. The blood pressure is measured by placing a cuff around the patient's arm and using a blood pressure gauge.

As the pulse rate and blood pressure measurements by nurses and other medical professionals are typically done with a measurement frequency of once an hour, it was difficult to respond to any abrupt changes in the conditions of dialysis patients. A simple way to solve this problem may be to conduct measurements of pulses and blood pressures more frequently, but it is not desirable as it would increase the burden of nurses and medical professionals.

Dialysis therapy is normally performed for four to five hours, during which time dialysis patients spend time by sleeping, watching TV, reading books, etc. These activities have to be interrupted by the measurements of pulses and that creates some stresses on the patients.

In order to eliminate these problems, various proposals have been made to measure the pulses and blood pressures of the patients automatically during the extracorporeal dialysis.

For example, Japanese Laid-open Patent Application No. 2002-186590 (JP'590) proposed a method of continuously measuring the pulse rate of a patient under the dialysis therapy by continuously measuring the deformation of the elastic tube between the dialyzer and the patient. Also, Japanese Laid-open Patent Application No. 2002-186665 (JP'665) proposed a method of measuring the blood pressure of a patient under extracorporeal dialysis by continuously measuring the deformation of the elastic tube between the dialyzer and the patient based on the same principle.

However, the method of measuring the pulse rate and the blood pressure by measuring the deformations of the elastic tube connecting the dialyzer and the patient can be affected by variations in the quality of the elasticity of the elastic tube, chronological changes in the elastic force of the tube, or fluctuations in the amount of deformation of the tube due to ambient temperature and humidity, thus compromising the accuracies of the measured values of pulses and blood pressures. Another problem is that there is a need for preparing a device specifically for accurately measuring the deformation of the elastic tube.

Moreover, the problems associated with the medical devices for transporting or circulating various fluids to or through the blood vessels of the patient includes problems related to the blood access in addition to problems associated with the measurements of pulse rates and blood pressures.

Dialysis devices, artificial heart-lung machines, or infusion pumps are known medical devices for transporting to or circulating through patients' blood vessels via blood vessel access. In case of a dialysis device, for example, the fluid that is transported is blood, and a system returns the blood back to the body after removing wastes accumulated in the blood by means of the dialysis device. This causes the blood to circulate. Also, a fusion pump is connected to the patient's blood vessel via a blood vessel access and a fluid medicine or a nutrient fluid is transported to the blood vessel. Thus, causing the fluid medicine or the nutrient fluid to be infused into the patient's body via the blood vessel access.

A typical dialysis device session is described below. First, a vascular cannula is inserted to provide a blood vessel access. The blood is then taken out of the patient's body through the blood vessel access by operating a blood pump and passes through the dialyzer. Waste substances and water in the blood are removed as a result of an osmotic pressure difference between the dialysate flowing outside of a hollow fiber provided in the dialyzer and the blood flowing inside of the hollow fiber as well as ultrafiltration. This process accomplishes the blood cleaning. The extracorporeal dialysis is normally performed for four to five hours, during which time dialysis patients spend time by sleeping, watching TV, reading books, etc.

There is a possibility of causing a serious accident if the vascular cannula disconnects from the blood vessel when the patient changes position during a sleep. Disconnection of a cannula on the vein side may cause a continuous loss of blood while disconnection of a cannula on the artery side may cause a danger of introducing air into the blood vessels. Either case creates a critical situation to the patient's safety. As a countermeasure, the prior art detects the disconnection of the cannula on the artery side by detecting air bubbles using an air detector connected to the artery side. The detector not only detects the disconnection of the cannula, but also a twisting of the blood vessel tube, the latter causing a poor circulation of the blood being dialyzed.

Japanese Laid-open Patent Application No. 011-513270 (JP'270) proposed a method for detecting the disconnection of a cannula used in dialysis. According to this method, the pressure wave generated by the heart is detected by a pressure sensor via the blood being dialyzed and it determines that the cannula is disconnected if no pressure wave is detected. A problem with this method is that the pressure waves applied to the blood being dialyzed consist not only of the pressure waves caused by the patient's heartbeat, but also include the pressure waves caused by the blood pump, so that the method leaves a task of how to extract only the pressure waves caused by the patient's heartbeat. The method of JP'270 uses a device of extracting only the pressure waves caused by the heart using a band-pass filter or a similar device taking advantage of the fact that the frequencies of the pressure waves generated by the heart and the pressure waves generated by a blood pump are different.

However, the frequency of the pressure waves of the patient's heart, i.e., pulse rate, is not constant but can change as the patient's condition changes. When the rotational frequencies of the blood pump and the pulse rate come close or overlap with each other, the device may eliminate not only the frequency caused by the blood pump but also the frequency caused by the pulse rate, and may end up being unable to detect the disconnection of the cannula used for the blood vessel access.

The present invention addresses the abovementioned situation, the first object of which is to provide, a method of constantly measuring pulse rates and a method of constantly measuring blood pressures without burdening patients or medical professionals, without having to add any special measuring device, and without being affected by the ambience such as the ambient temperature and humidity, as well as a medical device in which the above methods are applied.

The second object of the invention is to provide a method to securely monitor a blood vessel access used in a medical device even in a case where the patient's pulse rate changes substantially due to deterioration the patient's status resulting in overlapping of the frequency wave caused by the patient's heartbeat and the frequency of the pressure wave caused by the blood pump of the medical device and such, as well as a medical device using such a method.

### SUMMARY OF THE INVENTION

The invention relates to a method of measuring the pulse rate in a medical device, which is connected a patient's blood vessel by a blood vessel access and transports fluids to the blood vessel. The abovementioned object of the invention is achieved by measuring the pressure of the fluid. A frequency analysis is applied to the measurement data of the pressures for a certain period of time to obtain the spectrum consisting of various frequency components. The frequency components caused by mechanical devices (i.e. a pump) are removed from the spectrum to identify the frequency component caused by the patient's heartbeat. The pulse rate is measured from the frequency of the frequency component caused by the patient's heartbeat.

The abovementioned object of the invention is also achieved by measuring the pressure of the fluid while causing the pump to rotate with rotational frequencies varying, within a specific frequency range of the pump's basic frequencies respectively. The frequency analysis is applied to the measurement data of the pressures for a certain period of time to obtain the spectrum consisting of frequency components. The frequency components caused by the mechanical devices are removed from the spectrum to identify the frequency component caused by the patient's heartbeat. The pulse rate is measured from the frequency of the frequency component caused by the patient's heartbeat.

The invention relates to a method of measuring the blood pressure in a medical device connected a patient's blood vessel by a blood vessel access and has a mechanical device for applying a pressure to a fluid in order to transport the fluid to the blood vessel. The above-mentioned object of the invention is achieved by measuring the pressure of said fluid, applying a frequency analysis to the measurement data of the pressure for a certain period of time to obtain a spectrum consisting of frequency components, and removing the frequency components caused by the mechanical devices from the spectrum. The method identifies the frequency component caused by the patient's heartbeat, and the blood pressure can be measured from the intensity of the frequency component caused by the patient's heartbeat.

The abovementioned object of the invention is also achieved by measuring the pressure of the fluid while causing the pump (the mechanical device) to change its rotation frequency to vary within a specific frequency range around its basic frequency. A frequency analysis is applied to the measurement data of said pressure for a certain period of time to obtain a spectrum consisting of frequency components. The frequency components caused by the mechanical devices are removed from the spectrum to identify the frequency component caused by the patient's heartbeat, and the blood pressure can be measured from the intensity of the frequency component caused by the patient's heartbeat.

The invention relates to a medical device, which is connected a patient's blood vessel by a blood vessel access and has a mechanical device for applying a pressure to a fluid in order to transport the fluid to said blood vessel. The abovementioned object of the invention is achieved by including a pulse rate measuring circuit having a pressure detection device for measuring the pressure of said fluid; a frequency analysis device for applying a frequency analysis to the measurement data of the pressure for a certain period of time to obtain a spectrum consisting of frequency components; a removal device for removing the frequency components caused by said mechanical devices from the spectrum; and a pulse rate conversion device for converting the frequency of the frequency component caused by the patient's heartbeat into a pulse rate.

Moreover, the object of the invention is achieved by a blood pressure measuring circuit including a pressure detection device for measuring the pressure of the fluid; a frequency analysis device for applying a frequency analysis to the measurement data of the pressure for a certain period of time to obtain a spectrum consisting of frequency components; a removal device for removing the frequency components caused by the mechanical devices from the spectrum; and a blood pressure conversion device for converting the intensity of the frequency component caused by the patient's heartbeat into a patient's blood pressure. The invention relates to a method of monitoring a blood vessel access in a medical device connected a patient's blood vessel and having a mechanical device for applying a pressure to a fluid in order to transport the fluid to said blood vessel. The abovementioned object of the invention is achieved by measuring the pressure of said fluid, applying a frequency analysis to the measurement data for a certain period of time, obtaining a spectrum consisting of frequency components, removing the frequency components caused by the mechanical devices from the spectrum to identify the frequency component caused by the patient's heartbeat, and monitoring anomalies of the blood vessel access by judging the level of intensity of the frequency component caused by the patient's heartbeat.

The abovementioned object of the invention is also achieved by measuring the pressure of a fluid while causing the pump to change its rotation frequency to vary within a specific frequency range around its basic frequency and applying a frequency analysis to the pressure measurement for a certain period of time to obtain a spectrum consisting of frequency components. Next, removing the frequency components used by the mechanical devices from the spectrum to identify the frequency component caused by the patient's heartbeat, and monitoring anomalies in the blood vessel access by analyzing the level of intensity of the frequency component caused by the patient's heartbeat.

The abovementioned object of the invention is also achieved by measuring the pressure of the fluid, applying a frequency analysis to the pressure measurement data for a certain period of time, obtaining a spectrum consisting of frequency components, storing said spectrum as a first spectrum, storing the spectrum after a certain period of time as a second spectrum, taking a difference between the frequency components of the first spectrum and the frequency components of the second spectrum, and monitoring blood vessel access anomalies by making judgments on the level of intensity of the remaining frequency component.

The invention relates to a medical device, which is connected a patient's blood vessel by a blood vessel access and transports a fluid to the blood vessel. The abovementioned object of the invention can be achieved with a blood vessel access monitoring circuit having a pressure detection device for measuring the pressure of said fluid and a frequency analysis device for applying a frequency analysis to the pressure measurement data for a certain period of time to obtain a spectrum consisting of frequency components. A removal device removes the frequency components caused by a fluid transport device from said spectrum; and an analyzer for judging blood vessel access anomalies by measuring the level of intensity of the frequency component caused by the patient's heartbeat into a pulse rate.

The abovementioned object of the invention can also be achieved with a blood vessel access monitoring circuit including a pressure detection device for measuring the pressure of the fluid, and a frequency analysis device for detecting a spectrum consisting of frequency components by applying a frequency analysis device to the pressure measurement data for a certain period of time. A first storage device stores the spectrum as a first spectrum, a second storage device stores the spectrum after a certain period of time as a second spectrum, and then taking a difference between the frequency components of the first spectrum and the frequency components of the second spectrum. A judgment device analyzes the level of intensity of the remaining frequency component.

### BRIEF DESCRIPTION OF THE DRAWINGS

FIG. 1 is a diagram showing schematic version of a dialysis device of the present invention.

FIG. 2 is a diagram showing an arterial pressure waveform and a venous pressure waveform of the circulating blood of the dialysis device.

FIGS. 3(A) and 3(B) show spectral diagrams after FFT analyses of an arterial pressure waveform and a venous pressure waveform of the circulating blood of the dialysis device of FIG. 1.

FIG. 4 is a diagram showing a relation between the intensity of the frequency component caused by the patient's heartbeat and the blood pressure.

FIGS. 5A and 5B illustrate a pulse rate measuring circuit and a blood pressure measuring circuit according to an embodiment of the invention.

FIG. 6 is a diagram showing an embodiment of the invention for identifying the frequency component caused by the patient's heartbeat.

FIG. 7 is a diagram showing another embodiment for identifying the frequency component caused by the patient's heartbeat.

FIG. 8 is a diagram showing a further embodiment where the frequency component caused by the patient's heartbeat and the frequency components caused by the pump and such are overlapping.

FIG. 9 is a diagram showing a pulse rate display and a pulse rate alarm circuit of the present invention.

FIG. 10 illustrates a blood pressure display and a blood pressure alarm circuit of the present invention.

FIG. 11 is a diagram showing an embodiment in which a plurality of FFT analysis periods is used.

FIG. 12 is a schematic diagram illustrating the invention applied to a fluid infusion device.

FIG. 13 is a diagram showing arterial spectrums when the vascular cannula is disconnected and not disconnected.

FIG. 14 is a diagram showing a blood vessel access monitoring circuit applied to a dialysis device according to an embodiment of the invention.

FIG. 15 is a diagram showing an embodiment of a blood vessel access monitoring circuit where the frequency component caused by the patient's heartbeat and the frequency components caused by the pump overlap.

FIG. 16 is a diagram showing an embodiment of a blood vessel access monitoring circuit wherein the rotation frequency of the pump is changed.

FIG. 17 is a diagram showing an embodiment of a blood vessel access monitoring circuit according to the invention wherein blood vessel accesses are monitored by detecting the change of frequency spectrum distributions measured with a certain time interval.

FIG. 18 is a diagram showing an embodiment of a pressure detection device according to the present invention.

### DETAILED DESCRIPTION OF EMBODIMENTS OF THE INVENTION

The followings are the descriptions of embodiments of the invention, i.e., the method of measuring the patient's pulse rate, the method of measuring the blood pressure, and the medical device using said methods, as well as the second embodiment, i.e., the method of securely monitoring the blood vessel access, and the medical device using said method.

First, the method of measuring the patient's pulse rate, the method of measuring the blood pressure, and the medical device using said methods will be described below.

In case of a medical device having a mechanical device for transporting a fluid into the patient's blood vessel via a blood vessel access, a pressure wave applied to the fluid contains a pressure wave caused by the pump and such used for transporting the fluid and the pressure wave caused by the patient's heartbeat. For example, in case of a dialysis device, the circulating blood of the patient is the fluid, and the pressure waves according to the blood pump and the dialysate circulation pump exist in the blood in addition to the blood pressure wave that may be synchronized with the patient's heartbeat. Therefore, it is noted that if only the pressure wave caused by the patient's heartbeat can be identified from the mixture of the pressure waves and the frequency of said pressure wave can be measured, the patient's pulse rate can be measured. Also, since the intensity of the pressure wave can be measured, the patient's blood pressure can be measured.

An object of the present invention is to separate the weak pressure wave caused by the heartbeat existing in the blood circulating through the dialysis device by frequency analysis such as Fourier transformation, especially fast Fourier transformation (“FFT”), instead of using a band-pass filter as in the prior art. Attempting to separate the pressure wave caused by the patient's heartbeat using a conventional band-pass filter may be difficult when the pressure wave caused by a blood circulating pump and the pressure wave caused by the patient's heartbeat are too close or overlap with each other. The present invention can eliminate such a problem. An additional benefit here is that the FFT analysis is more robust than the band-pass filter method against irregular motions. The FFT analysis reacts on repetitive phenomena but it does not react on irregular movements such as the patient's body motion.

In the present invention, the frequency analysis is applied to the pressure wave applied on the blood circulating through the dialysis device to detect a spectrum consisting of various frequency components, and separate the frequency component caused by the patient's heartbeat from the frequency components caused by the blood pump.

The patient's pulse rate can be measured as the frequency component of the pressure wave caused by the patient's heartbeat. The patient's blood pressure can be measured as the intensity of the frequency component of the pressure wave caused by the patient's heartbeat.

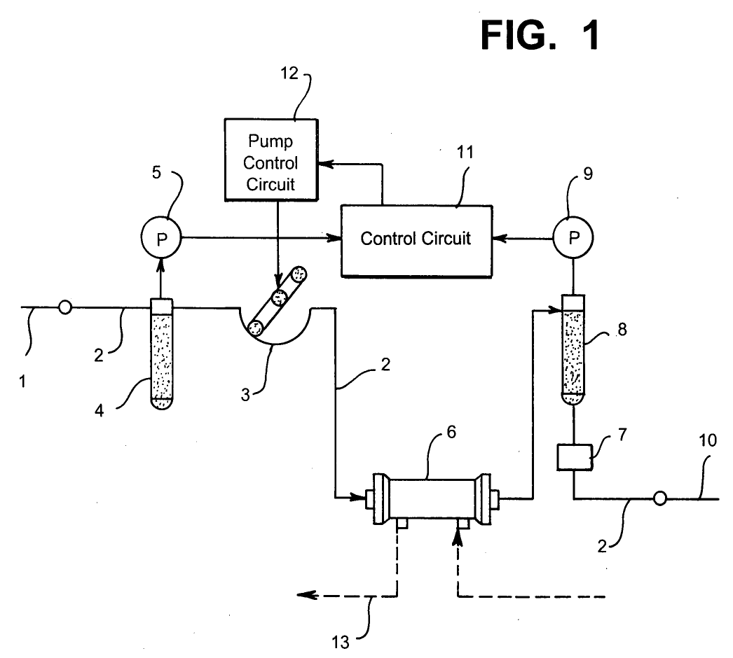

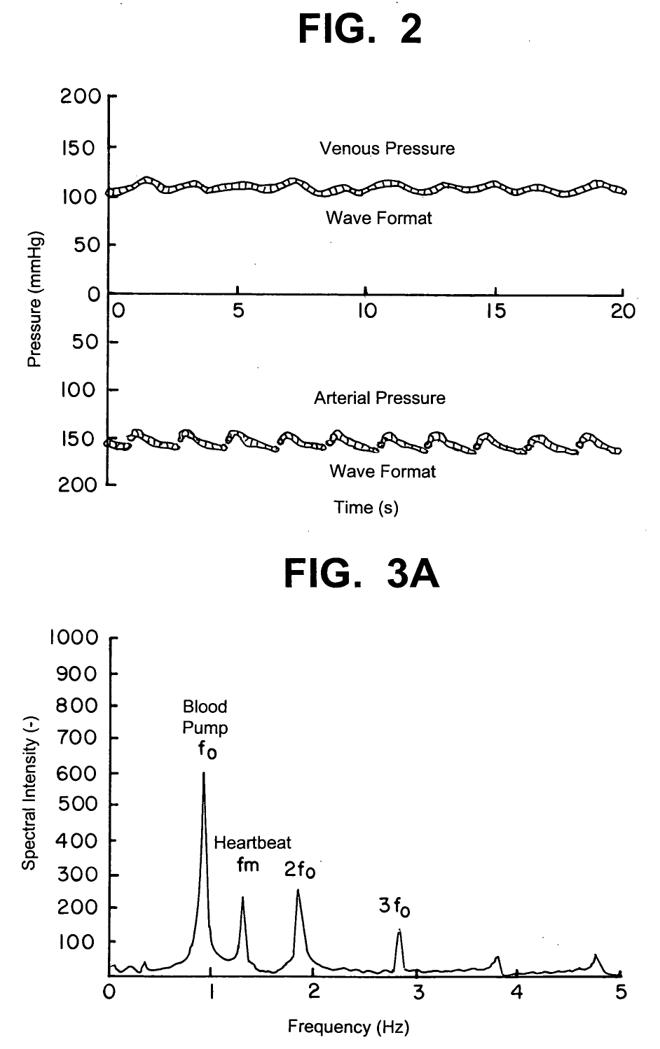

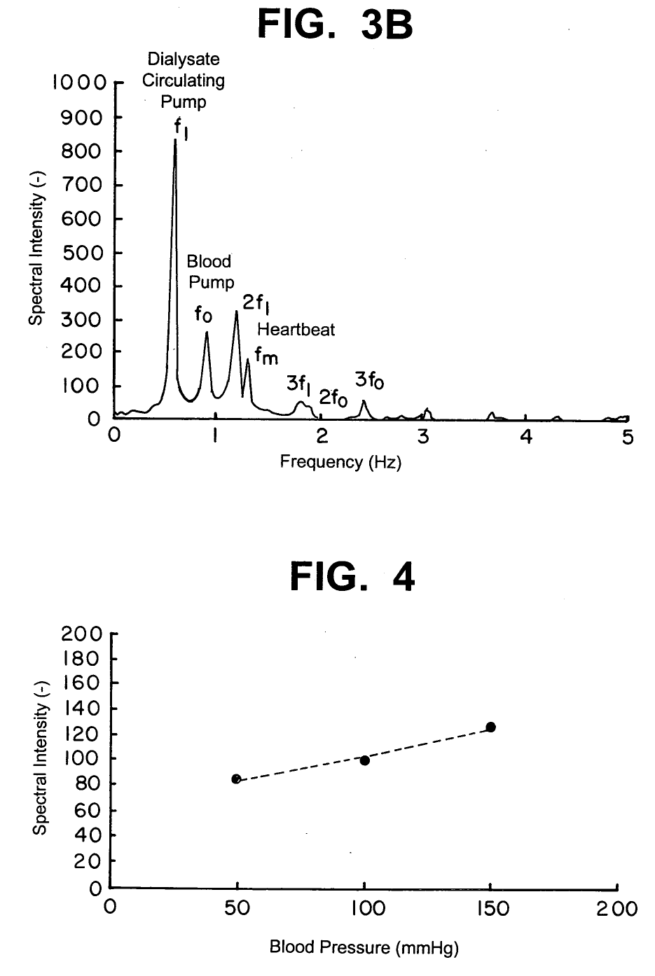

Below is a discription of a dialysis device and about the mixture of pressure waves existing in the blood circulating through the dialysis device so that the invention can be understood more easily. FIG. 1 shows a schematic dialysis device and FIG. 2 is an arterial pressure waveform and a venous pressure waveform of the circulating blood. FIG. 3(A) shows the spectrum of the arterial pressure waveform of the circulating blood after the FFT analysis, and FIG. 3(B) shows the spectrum of the venous pressure waveform of the circulating blood after the FFT analysis.

In FIG. 1, the patient's blood being dialyzed is circulated forcibly by a blood pump 3 through an arterial cannula 1 inserted into the patient's blood vessel and a blood tube 2, and transported to a dialyzer 6 via an arterial drip chamber 4. After wastes contained in the patient's blood are filtered in a dialyzer 6, the patient's blood is transported to a venous drip chamber 8 and then returned to the patient's blood vein through venous blood tube 2 and a venous cannula 10. The wastes removed from the blood move to the dialysate in dialyzer 6 and the dialysate containing the wastes is transported through a dialyzing tube 13.

The pressures being applied to the circulating blood include the pressure caused by blood pump 3, which is the largest, as well as the pressure according to the dialysate circulation pump (not shown) and the pressure caused by the patient's heartbeat. An artery pressure sensor 5 and a venous pressure sensor 9 are provided to detect these pressures applied to the circulating blood. Although either the arterial pressure data obtained by arterial pressure sensor 5 or the venous pressure data obtained by venous pressure sensor 9 can be used for the invention, it is easier to identify the frequency component caused by the patient's heartbeat if the arterial pressure data is used.

The arterial pressure data of the circulating blood obtained by arterial pressure sensor 5 is thus sent to a control circuit 11 of the dialysis device. A pump control circuit 12 is capable of operating blood pump 3 based on the rotation frequency instructed by control circuit 11 as well as detecting the rotation frequency of blood pump 3 and transmitting the detected rotation frequency to control circuit 11.

FIG. 2 shows the pressure wave format data of the circulating blood observed by arterial pressure sensor 5 and venous pressure sensor 9, wherein the output data of arterial pressure sensor 5 is shown on the negative side of FIG. 2 and the output data of venous pressure sensor 9 is shown on the positive side of FIG. 2.

FIGS. 3(A) and 3(B) show spectral diagrams after FFT analyses of these pressure waveforms. FIG. 3(A) shows the spectral diagram of the arterial pressure waveform of the circulating blood after the FFT analysis, and FIG. 3(B) shows the spectral diagram of the venous pressure waveform of the circulating blood after the FFT analysis. Thus, the pressure waveform data shown in FIG. 2 contains the frequency components shown in FIG. 3(A) and FIG. 3(B), and the spectral diagrams such as shown in FIG. 3(A) and FIG. 3(B) can be obtained by applying FFT analysis to pressure wave data. This is the key point to the present invention, because it reveals the fact that the spectral diagrams consisting of various frequency components such as FIG. 3(A) and FIG. 3(B) can be obtained by applying the FFT analysis to the frequency component caused by a patient's heartbeat, which is completely invisible in the pressure waveform shown in FIG. 2.

The venous spectrum shown in FIG. 3(B) contains a mixture of the frequency component of frequency f₀ caused by blood pump 3 and the frequency component of frequency f₁ caused by the dialysate circulation pump in addition to the frequency component of frequency fₘ caused by the patient's heartbeat. Moreover, other frequency components caused by the pumps such as frequencies 2f₀, 3f₀, and 2f₁, which are integral multiples of the basic frequencies of the pumps, f₀ and f₁ respectively, exist in the mixture. Therefore, the task is how to identify only the frequency component caused by the patient's heartbeat from the spectrum consisting of a mixture of various frequency components.

The invention provides several methods for identifying the frequency components caused by the pressures of the mechanical devices such as pumps applied on the blood from the mixture of frequency components existing in the circulating blood.

An embodiment enables detecting only the frequency components of the pressure waves caused by blood pump 3 and the dialysate circulation pump by operating the mechanical devices, such as blood pump 3 and the dialysate circulation pump prior to the installation of blood vessel accesses such as arterial cannula 1 and venous cannula 10. Once the data of the frequency components caused by the mechanical devices are obtained and stored into the storage device of the control circuit, there is no need to obtain them each time when a dialysis is performed until the mechanical devices of the dialysis device are replaced or deteriorated.

Another embodiment measures the patient's pulse rate from the frequency components resulting from removing the frequency components caused by the rotations of the pumps such as blood pump and the dialysate circulation pump from the spectrum after the FFT analysis. The rotation frequencies of those pumps are already known to control 11 and pump control circuit 12.

A further embodiment is blood pump 3 and the dialysate circulation pump by fluctuating their rotational frequencies within a certain range of the standard frequency respectively suited for the patient's dialysis condition and does not burden the patient while executing dialysis with arterial cannula 1 and venous cannula 10 properly installed. The FFT analysis is used because it detects only those frequency components that appear repeatedly at exact same frequencies and does not detect frequency components whose frequencies are constantly changing. Therefore, the frequency components caused by the pumps will not be detected by the frequency analysis device if the pumps are operated by fluctuating their rotation frequencies within a certain range. Thus, only the frequency component of the pressure wave caused by the patient's heartbeat will appear in the output of the frequency analysis device. When the rotation frequency of a pump is fluctuated within a certain range, in particular, with a frequency synchronizing with the sampling frequency of the FFT, the frequency component caused by said pump can be removed more efficiently than when it is not synchronized with the sampling frequency.

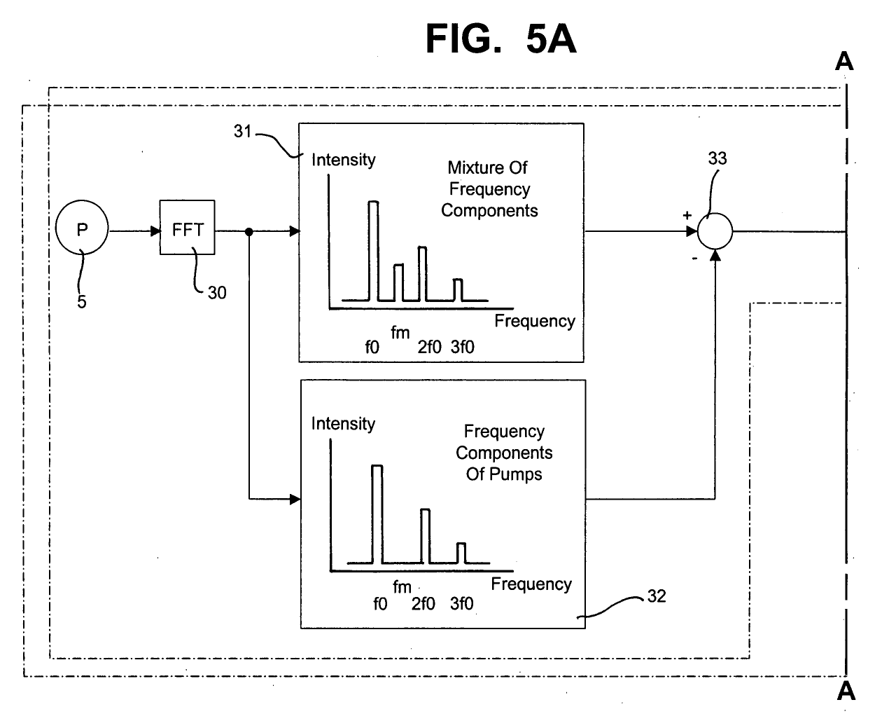

The patient's blood pressure and pulse rate can be calculated from the frequency component of the frequency caused by the pulse rate, when it can be identified. The calculation can be performed because the intensity of the frequency component caused by the pulse rate and the blood pressure are in a generally proportional relationship, as shown in FIG. 4. In FIG. 4, the vertical axis represents the intensity of the frequency component and the horizontal axis represents the blood pressure. FIG. 4 illustrates that the blood pressure can be estimated from the intensity of the frequency component caused by the pulse rate using this relation.

The outline of the embodiment procedures of the present invention will be described below based on the basic principles described above.

The method includes measuring a spectrum of all the frequency components containing the pressure waves caused by the heartbeat, and the pumps by analyzing the pressure waves being applied on the circulating blood with the FFT analysis.

A step is identifying the frequency components of the pressure wave caused by the mechanical devices that affect the pressure of the circulating fluid, such as the blood pump and the dialysate circulation pump, other than the patient's heartbeat. There are several methods of identifying the frequency components of the pressure waves that are caused by the mechanical devices as described above.

Another step is removing the frequency components caused by the mechanical devices that are identified above from the spectrum having a mixture of all kinds of frequency components previously obtained, and to identify the resultant frequency component as the frequency component caused by the patient's heartbeat.

The patient's pulse rate can be calculated from the frequency component caused by the patient's heartbeat. The blood pressure can be calculated from the intensity of the frequency component caused by the patient's heartbeat.

The above is the outline of the working procedure of the present invention and a preferable embodiment of the invention will be described in more details referring to the accompanying drawings.

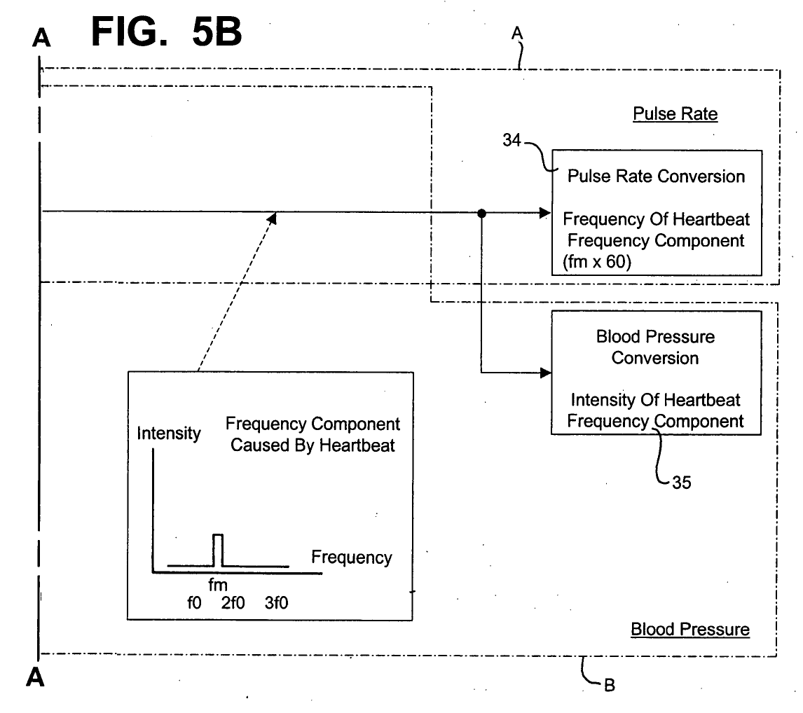

FIGS. 5A and 5B are diagrams illustrating a pulse rate measuring circuit and a blood pressure measuring circuit according to an embodiment of the invention. The portion surrounded by a single-dot chain line A corresponds to the pulse rate measuring circuit, and the portion surrounded by a double-dot chain line B corresponds to the blood pressure measuring circuit. Although the pulse rate measuring circuit A and the blood pressure measuring circuit B can be realized as either a hardware system or a software system, control circuit 11 consists of a microcomputer, so that the pulse rate measuring circuit A and the blood pressure measuring circuit B can be configured by a software system using the microcomputer, making it unnecessary to add any hardware allowing for a low cost and economic alternative to the prior art.

FIGS. 5A and 5B illustrate frequency analysis device 30 receiving as an input the pressure waveform data of the circulating blood obtained by the pressure detection device, i.e., arterial pressure sensor 5. Frequency analysis device 30 can be realized by a software program using a microcomputer, or by a hardware device such as a dedicated IC for FFT analysis. Although the use of an IC device is disadvantageous from a cost standpoint, it provides advantages such that its analysis speed is fast and it does not burden the microcomputer's CPU. If the FFT analysis is handled by a software program, there is no need for adding hardware device to implement this invention, so that it may not increase the device cost and/or alter the external shape of the device.

A certain time period can be set for the FFT analysis, frequency analysis device 30 sets up a certain time period for the FFT analysis. In FIG. 2, for example, the FFT is applied for a period of 0 to 5 seconds. The longer the time period, the more repetitions of waveform are entered for the FFT analysis.

Frequency analysis device 30 can output to a storage device 31. Storage device 31 stores the latest spectrum containing a mixture of frequency components caused by the mechanical devices, such as blood pump 3 and the dialysate circulation pump and the frequency component caused by the patient's heartbeat obtained by applying the FFT analysis by frequency analysis device 30 to the pressure wave data detected by arterial pressure sensor 5. The contents of storage device 31 are constantly updated with new data while the dialysis continues. Since storage 31 can optionally temporarily store the output data of frequency analysis device 30, storage device 31 can be built into frequency analysis device 30.

Further, the removing device includes a second storage device 32 and a subtraction device 33. Second storage device 32 stores the spectrum of the frequency components of the pressures applied to the blood caused by the mechanical devices, such as blood pump 3 and the dialysate circulation pump, obtained by the FFT analysis using frequency analysis device 30 from the pressure wave data detected by arterial sensor 5 prior to installing arterial cannula 1 and venous cannula 10 to the blood vessel, i.e., prior to installing blood vessel accesses.

After the data is collected prior to the start of the dialysis and prior to the installation of arterial cannula 1 and venous cannula 10 to the blood vessel, i.e., prior to the installation of blood vessel accesses, the data is stored in second storage device 32 and the same data is used during the dialysis process. Once the data of the frequency components due to the mechanical devices are obtained and stored in storage device 32 of the control circuit, as mentioned above, there is no need to obtain the data each time when a dialysis is performed until the mechanical devices of the dialysis device are replaced or deteriorated.

Subtraction device 33, being connected to storage device 31 and second storage device 32, is capable of subtracting the frequency components caused by only the mechanical devices such as blood pump 3 and the dialysate circulation pump, which are stored in second storage device 32, from the spectrum containing a mixture of the frequency components caused by the mechanical devices, such as blood pump 3 and the dialysate circulation pump, and the frequency component caused by the patient's heartbeat, which are stored in storage device 31, to obtain only the frequency component caused by the patient's heartbeat. The portion of the system configuration for identifying only the frequency component caused by the patient's heartbeat described so far is a part common to both the pulse rate measuring circuit A and the blood pressure measuring circuit B.

Pulse rate measuring circuit A detects the pulse rate from the frequency element fₘ of the frequency component caused by the patient's heartbeat obtained by subtraction device 33 by a pulse rate conversion device 34 is connected to the output side of subtraction device 33. Since the frequency of the spectrum obtained by the FFT analysis is measured in cycle/second, pulse rate conversion device 34 converts the output of subtraction device 33 into cycle per minute. In other words, the desired pulse rate can be achieved by multiplying fₘ by 60 (seconds/minute).

Blood pressure measuring circuit B can detect the blood pressure from the intensity of the frequency component caused by the patient's heartbeat obtained by subtraction device 33 by a blood pressure conversion 35 connected to the output side of subtraction device 33. As shown in FIG. 4, there is a certain relation between the intensity of the frequency component caused by the patient's heartbeat and the blood pressure. In FIG. 4, the vertical axis represents the intensity of the frequency component, which is substantially proportional to the blood pressure represented by the horizontal axis. In this embodiment, the function of blood pressure conversion device 35 is described as being a proportional relation, but a separate conversion table based on a precise measurement can be used instead to represent the relation between the intensity of the frequency component and the blood pressure.

The above embodiment shows a method of measuring the frequency components caused by the pumps prior to the installation of the blood vessel accesses as a method of obtaining only the frequency component caused by the patient's heartbeat by subtracting the frequency components caused by blood pump 3 and the dialysate circulation pump from the spectrum containing a mixture of the frequency components caused by the mechanical devices such as blood pump 3 and the dialysate circulation pump and the frequency component caused by the patient's heartbeat.

Next, for another embodiment, the operating frequencies of blood pump 3 and the dialysate circulation pump, f₀ and f₁, are already known to control circuit 11 and pump control circuit 12. In other words, in FIG. 1, since control circuit 11 provides instructions to pump control circuit 12 about the rotation frequencies of blood pump 3 and the dialysate circulation pump, the rotation frequencies f₀ and f₁ are obviously known by control circuit 11. Therefore, masking the spectrum consisting of a mixture of frequency components existing in storage device 31 with the components of frequencies f₀, f₁ and their respective integral multiples leaves only the frequency component of frequency fₘ caused by the patient's heartbeat, and the frequency component of the frequency fₘ caused by the patient's heartbeat alone can be measured.

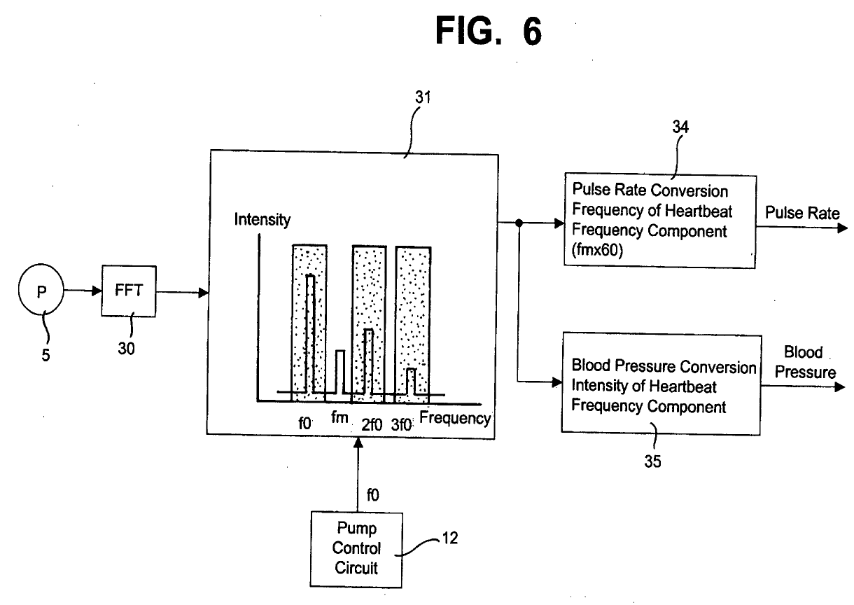

A removal device masks the latest spectrum containing a mixture of the frequency component caused by the patient's heartbeat and the frequency components caused by the pumps, which is supplied as an output of frequency analysis device 30 and stored in storage device 31, with the frequency components corresponding to the rotation frequencies of the pumps as shown in FIG. 6. Pump control circuit 12 can find the rotation frequencies f₀ and f₁ of blood pump 3 and the dialysate circulation pump from the operational instructions issued by control circuit 11 to pump control circuit 12, or more accurate frequencies than the operational instruction values by providing rotational speed sensors on blood pump 3 and the dialysate circulation pump in pump control circuit 12 if it is desired to use more accurate operation frequencies.

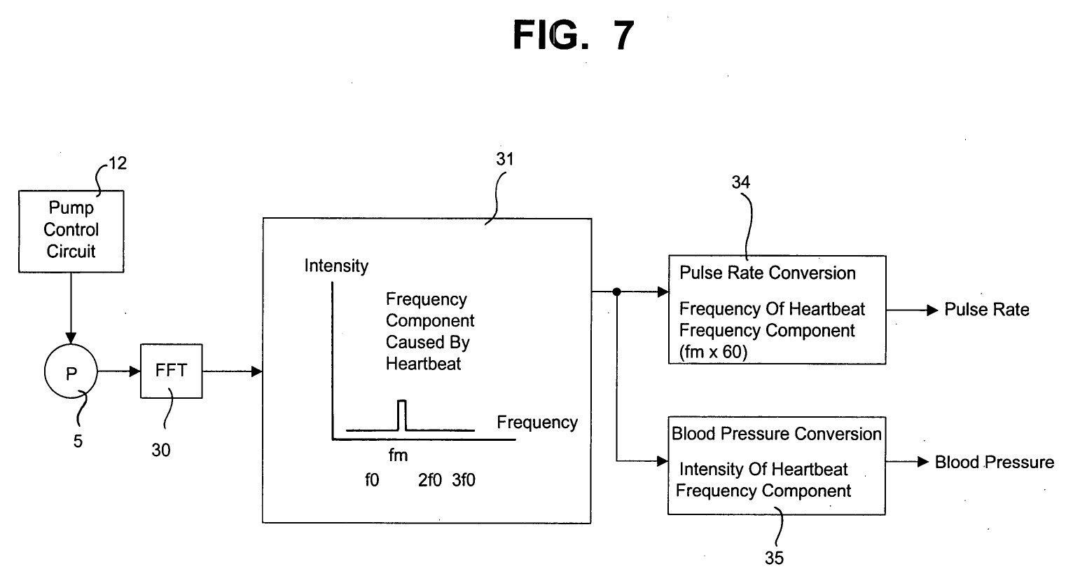

A further embodiment is described below referring to an embodiment shown in FIG. 7. This is a method based on control circuit 11 issuing a control instruction to pump control circuit in order to operate blood pump 3 and the dialysate circulation pump by fluctuating their rotational frequencies within a certain range of the standard frequency respectively suited for the patient's dialysis condition that does not burden the patient while executing dialysis with arterial cannula 1 and venous cannula 10 properly installed. The FFT analysis is used here because of its characteristic that the FFT detects only those frequency components that appear repeatedly at exact same frequencies and does not detect frequency components whose frequencies are constantly changing. As a result, only the frequency component caused by the heartbeat appears at storage device 31 in this case, enabling identification of frequency component caused by the heartbeat. The output from storage device 31 is thus entered directly into pulse rate conversion device 34 and blood pressure conversion device 35.

When the rotation frequencies of blood pump 3 and the dialysate circulation pump are fluctuated within a certain range respectively, in particular, with a frequency synchronizing with the sampling frequency of the FFT, the frequency components caused by said pumps can be removed more efficiently than when they are not synchronized with the sampling frequency.

Since the pulse rate can be determined from the frequency fₘ of the frequency component caused by the pulse rate, the detection of the frequency, not the intensity of the frequency component, is considered sufficient for measuring the pulse rate. However, even when the rotation frequencies of blood pump 3 and the dialysate circulation pump are to close to or overlap the patient's pulse rate due to a change in the pulse rate, the frequency components caused by the pumps and the frequency component caused by the pulse rate can be separated if the intensities are considered in addition to the frequencies of the frequency components.

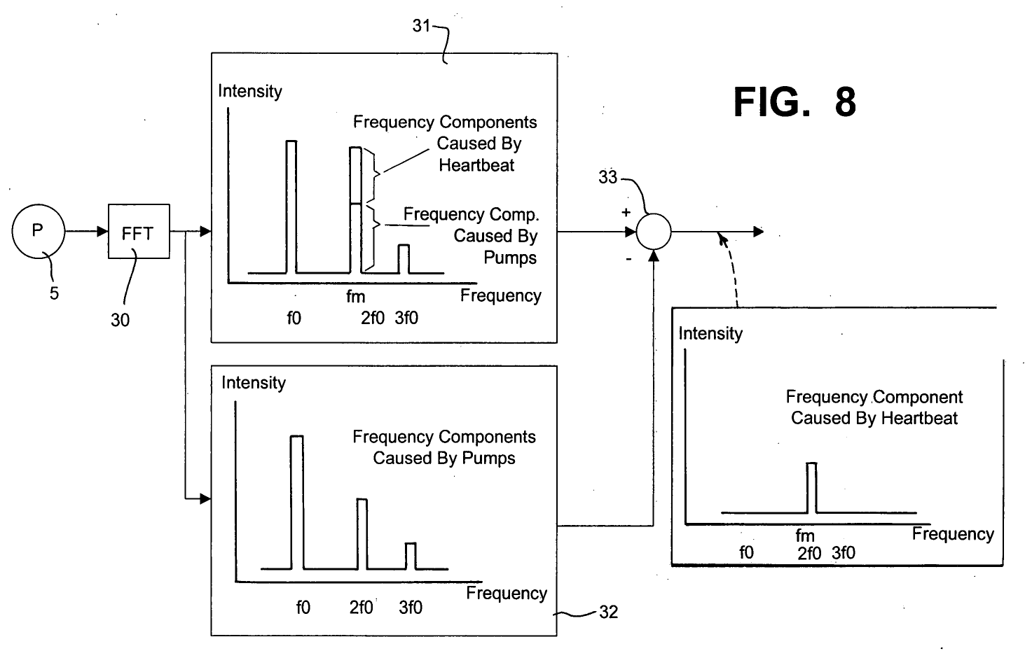

FIG. 8 shows an embodiment where the frequency component caused by the heartbeat is identified by considering the intensity of the frequency component. Since the intensity stored in second storage device 31 is that of the frequency component, which is the sum of the frequency component caused by the patient's heartbeat and the frequency components caused by the pumps, subtracting the intensity of the frequency components caused by the pumps stored in storage device 32 from the intensity stored in storage device 31 by subtraction device 33 produces the intensity of the frequency component caused by the heartbeat alone as a remainder, and the pulse rate can be determined from the remainder. Thus, the frequency component caused by the heartbeat can be identified to allow the determination of the pulse rate in this embodiment even when both kinds of frequencies are overlapping and their separation is difficult with a band-pass filter.

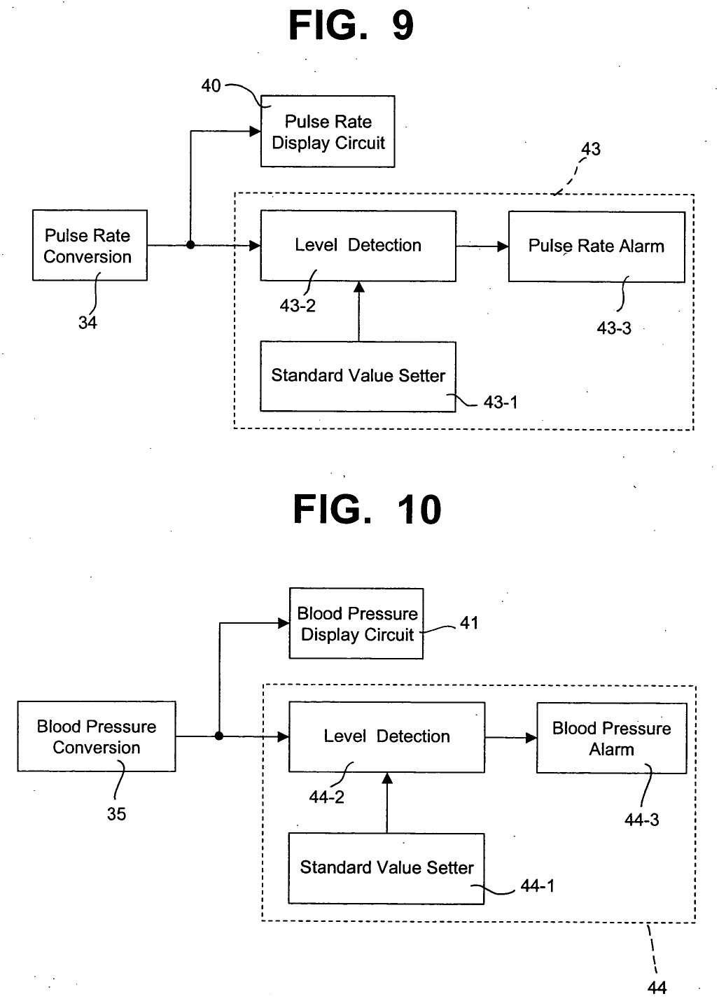

Next, in order for medical professionals or the patient to know the pulse rate and the blood pressure, a pulse rate display 40 and a blood pressure display 41 for displaying the patient's measured pulse rate and blood pressure respectively are provided on the dialysis device as indicated in FIG. 9 and FIG. 10. These displays can be realized either by a digital display or an analog display and using these displays, medical professionals can quickly ascertain the patient's status.

A pulse rate warning circuit 43, and/or a blood pressure warning circuit 44 can be added to the dialysis device in order to warn medical professionals quickly when the pulse rate and the blood pressure are abnormal as shown in FIG. 9 and FIG. 10 wherein the outputs of pulse rate conversion 34 and blood pressure conversion 35 are compared with the normal pulse rate and the normal blood pressure of standard value setters 43-1 and 44-1 at level detectors 43-2 and 44-2 respectively. Since the values of standard value setter 43-1 for the normal pulse rate and standard value setter 44-1 for the normal blood pressure vary with each patient, a setting unit such as a key-board can be provided in control circuit 11 in order to set them up in each case. For example, the high blood pressure threshold can be set at 200 mm Hg and the low blood pressure threshold at 50 mm Hg for the blood pressure, or set the tachycardia threshold at 150 cycles/minute for the pulse rate.

Although we have been describing the time period for applying the FFT analysis (FFT analysis period) limited to one case (t₀−t₁) up to this point, a patient's status can be more closely monitored by setting up a plurality of FFT analysis periods. In other words, although the pulse rate and the blood pressure can be calculated more accurately by taking a longer FFT analysis period (e.g., t₀−t₁=5 sec), the FFT analysis period should be set shorter (e.g., t₀−t₂=1sec), if speed is more critical than accuracy in order for the device to be able to take a quick action when an anomaly occurs with the patient's status. Consequently, a more patient-oriented dialysis service can be provided by setting up a plurality of FFT analysis periods, displaying the pulse rate and the blood pressure corresponding to each analysis period on the pulse rate display and the blood pressure display, using that data for the pulse rate alarm circuit and the blood pressure alarm circuit in order to satisfy those conflicting demands.

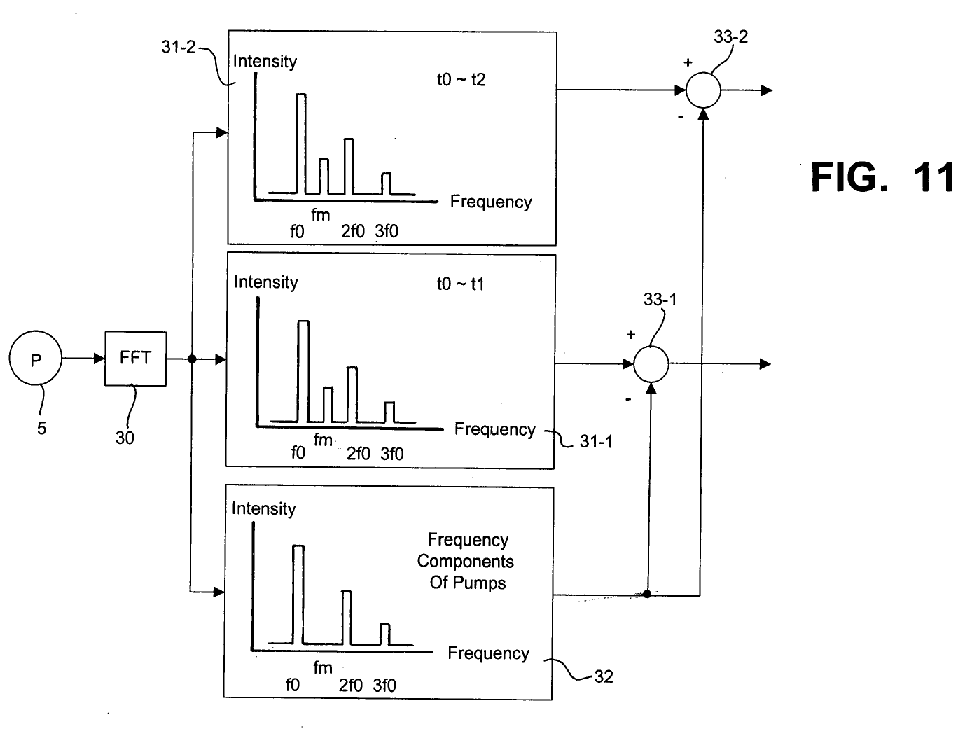

FIG. 11 shows an embodiment based on such a concept. A plurality of storage devices 31, 31-1 and 31-2, corresponding with the analysis periods if there are two kinds of FFT analysis periods (t₀−t₁=5 sec and t₀−t₂₌₁ sec). Alternately, an analysis instruction is issued to FFT analysis 30 with two kinds of FFT analysis periods, t₀−t₁ and t₀−t₂, and the analysis result of t₁−t₁ is stored in storage device 31-1, while the analysis result of t₀−t₂ is stored in storage device 31-2.

The storage data of second storage device 32 are the frequency components of the pumps measured once prior to the installation of the vascular cannula at the start of a dialysis operation, and the same data can be used throughout the dialysis operation unless the operating frequencies of the pumps are changed.

The pulse rate and the blood pressure obtained from storage device 31-1 storing spectrums intensity for longer FFT analysis periods, i.e., with higher accuracies, are used for pulse rate display 40 and blood pressure display 41. Alternately, the pulse rate and the blood pressure obtained from storage device 31-2 storing spectrums with higher detection speeds although of lesser accuracies, can be used for pulse rate display 43 and blood pressure display 44 to provide closer monitoring of the patient's status. It is possible to use multiple setup periods instead of two kinds of setup periods.

Although a case of detecting the circulating blood with arterial pressure sensor 5 has been described so far, it is also possible to measure both the pulse rate and the blood pressure from the spectrum shown in FIG. 3(B) using an embodiment shown in FIG. 5 even when the blood is detected with venous pressure sensor 9.

Also, using the FFT analysis in this embodiment provides an excellent feature that the patient's pulse rate and pressure blood can be measured more securely as it prevents the analyzer's malfunction which might otherwise be caused by pressure waves applied to the blood as a result of irregular motions of the patient as in a case when the patient turns over in bed.

Moreover, although it has been described so far about a case wherein the FFT analysis is used as the frequency analysis for separating the frequency component of the pressure wave caused by the heartbeat from the frequency components of the pressure wave caused by the mechanical devices such as the pumps, it is known that other frequency analysis device are capable of differentiating the frequencies of these pressure waves other than the FFT analysis, for example, the normal Fourier analysis and the MEM (maximum entropy method), can be used for the same purpose.

The present invention can be applied not only to the measurement of the patient's pulse rate and blood pressure in a dialysis device, but also to the measurement of the patient's pulse rate and blood pressure in medical devices such as infusion pump devices and artificial heart-lung machines.

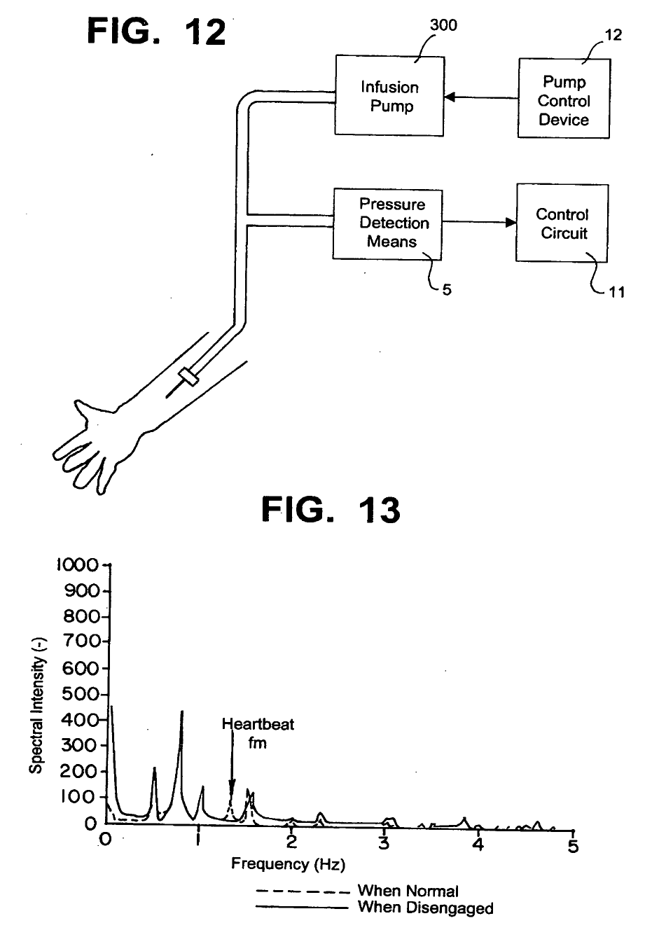

FIG. 12 is an embodiment of the invention applied to a fluid infusion device. A fluid infusion device is different from a dialysis device in that it is neither for circulating blood nor for measuring the blood pressure of circulating blood. A fluid infusion device, infuses a solution into a blood vessel, the pressure applied to the infusion fluid is detected by pressure detecting device 5, and a mixture of frequency component of the pressure wave caused by infusion pump 300 and a frequency component of the pressure wave caused by the heartbeat exists, so that it is possible to extract only the frequency component of the pressure wave caused by the heartbeat from said mixture in order to measure the patient's pulse rate and blood pressure.

Therefore, the present invention can be applied to dialysis devices, artificial heart-lung machines, infusion devices, or blood transfusion devices, so that it provides the benefits of accurately measuring the pulse rates and blood pressures of the patients being treated with these medical devices without burdening the patients or medical professionals, at any time, and without needing any additional devices.

As can be seen from the above description, the present invention has an excellent advantage of providing a method of accurately and continuously measuring the pulse rate and blood pressure of a patient being treated with a medical device connected to the patient's blood vessel through a blood vessel access and has a mechanical device for applying a pressure to transport a fluid to said blood vessel. The method does not burden the patient or medical professionals and does not require any additional equipment.

A theory and the embodiments of a secure method of monitoring blood vessel accesses and medical devices based on said method are described below.

In the present invention, it is necessary to identify only the pressure wave caused by the heartbeat from a mixture of pressure waves including the pressure wave caused by a pump used for transporting the fluid and the pressure wave caused by the blood pressure synchronized with the patient's heartbeat. The invention measures the intensity of said pressure wave caused by the heartbeat and makes a judgment that an anomaly exists in the blood vessel access to the patient. The judgement is based on if the intensity is abnormally weak or no pressure wave caused by the heartbeat exists. In other words, since the intensity of said pressure wave is proportional to the patient's blood pressure, it is assumed to be caused by either disconnection of the cannula of the patient's blood vessel access or twisting of the blood tube preventing the transport of the fluid, rather than hypotension, if the intensity of the pressure wave appears extremely lower than that can be caused by hypotension.

An embodiment separates the weak pressure wave caused by the heartbeat existing in the fluid being transported between the medical device and the patient by device of a frequency analysis such as Fourier transformation, especially fast Fourier transformation (“FFT”), instead of using a band-pass filter as in the prior art. If it is attempted to separate the pressure wave due to the heartbeat using a conventional band-pass filter, the separation may be difficult when the pressure wave caused by a blood pump as such and the pressure wave due to the heartbeat are too close or overlap with each other, but the present invention can eliminate such a problem. An additional benefit here is that the FFT analysis is more robust than the band-pass filter method against irregular motions, as the FFT analysis reacts on repetitive phenomena but it does not react on irregular movements such as the patient's body motion.

In the present invention, the frequency analysis is applied to the pressure wave applied on the fluid being transported between the medical device and the patient to detect a spectrum consisting of various frequency components, and separate the frequency component due to the heartbeat from the frequency component due to the blood pump and such.

First, the principle of the present invention when it is applied to a dialysis device of FIG. 1, and reffering to FIG. 2, FIG. 3, and FIG. 13. As described in the above, the fluid being transported in case of a dialysis device is the patient's blood. FIG. 1 shows the constitution of a dialysis device and FIG. 2 is an arterial pressure waveform and a venous pressure waveform of the blood circulating through the dialysis device. FIG. 3(A) and FIG. 3(B) show the spectrums of the arterial pressure waveform and of the venous pressure waveform of the blood circulating through the dialysis device respectively after the FFT analysis. FIG. 13 is a diagram showing arterial side spectrum of a case when the vascular cannula is attached being laid over the spectrum of a case when it is disconnected.

In FIG. 1, the fluid being transported through the blood vessel access is blood before dialysis containing wastes, or blood after dialysis. In such a dialysis device, the patient's blood to be dialyzed is transported from an arterial cannula 1 inserted into the patient's arterial side blood vessel through a blood tube 2 and an arterial drip chamber 4 into a dialyzer 6. After wastes contained in the patient's blood are filtered in a dialyzer 6, the patient's blood, removed of wastes, is transported to a venous drip chamber 8 and then returned to the patient's blood vein through arterial blood tube 2 and a venous cannula 10. This forced circulation of the blood is done by a blood pump 3. The wastes removed from the blood move to the dialyzing fluid in dialyzer 6 and the dialysate containing the wastes is transported through a dialyzing tube 13.

The pressures applied to the blood circulating the extracorporeal circulation circuit include the pressure caused by blood pump 3, which is the largest, as well as the pressure according to the dialysis device circulation pump and the pressure due to the patient's heartbeat. An artery pressure sensor 5 and a venous pressure sensor 9 are provided as device of detecting these pressures applied to the blood circulating the extracorporeal circulation circuit.

The arterial pressure data of the circulating blood obtained by arterial pressure sensor 5 is thus sent to a control circuit 11 of the dialysis device. A pump control circuit 12 is cable of operating blood pump 3 based on the rotation frequency instructed by control circuit 11 as well as detecting the rotation frequency of blood pump 3 and transmitting the detected rotation frequency to control circuit 11.

FIG. 2 shows the pressure wave format data of the circulating blood observed by arterial pressure sensor 5 and venous pressure sensor 9, wherein the output data of arterial pressure sensor 5 is shown on the negative side of FIG. 2 and the output data of venous pressure sensor 9 is shown on the positive side of FIG. 2.

FIGS. 3(A) and 3(B) show spectrum diagrams after FFT analyses of these arterial pressure waveforms. FIG. 3(A) shows the spectral diagram of the arterial pressure waveform of the circulating blood after the FFT analysis, and FIG. 3(B) shows the spectral diagram of the venous pressure waveform of the circulating blood after the FFT analysis. This device that the pressure waveform data shown in FIG. 2 contains the spectral data shown in FIG. 3(A) and FIG. 3(B), and the spectral diagrams such as shown in FIG. 3(A) and FIG. 3(B) can be obtained by applying the FFT analysis to the pressure wave data. This point of the present invention reveals the fact that the spectral diagrams consisting of various frequency components such as FIG. 3(A) and FIG. 3(B) can be obtained by applying the FFT analysis to the frequency component caused by the heartbeat, which is completely invisible in the pressure waveform shown in FIG. 2.

FIG. 13 is a diagram showing an arterial frequency spectrum of a case when the vascular cannula is attached being laid over the spectrum of a case when it is disconnected. Note that only the frequency component caused by the heartbeat disappears when the vascular cannula is disconnected. The disconnection of vascular cannula can be detected using this feature.

However, the question here is how to extract only the frequency component caused by the heartbeat from a mixture of the frequency components caused by the mechanical devices such as pumps and the frequency component caused by the heartbeat. The venous spectrum shown in FIG. 3(B) contains a mixture of the frequency component of frequency f₀ due to blood pump 3 and the frequency component of frequency f₁ due to the dialysate circulation pump in addition to the frequency component of frequency fₘ due to the heartbeat. Moreover, other frequency components caused by the pumps such as frequencies 2f₀, 3f₀, and 2f₁, which are integral multiples of the basic frequencies of the pumps, f₀ and f₁ respectively, exist in the mixture. Therefore, the task is how to identify only the frequency component due to the heartbeat from the mixture of various frequency components.

The invention provides several methods for identifying the frequency components due to the pressures of the mechanical devices such as pumps applied on the blood from the mixture of frequency components existing in the circulating blood.

A method enables detecting of only the frequency components of the pressure waves due to blood pump 3 and the dialysate circulation pump by operating the mechanical devices such as blood pump 3 and the dialysate circulation pump prior to the installation of blood vessel accesses such as arterial cannula 1 and venous cannula 10. Once the spectral data of the frequency components due to the mechanical devices are obtained and stored to the storage device of the control circuit, there is no need to obtain the data each time when a dialysis is performed until the mechanical devices of the dialysis device are replaced or deteriorated.

Another embodiment is based on operating blood pump 3 and the dialysate circulation pump by changing the frequency within a certain range of the basic frequency suited for the patient's dialysis condition that does not burden the patient while executing dialysis with arterial cannula 1 and venous cannula 10 properly installed. The FFT analysis uses a characteristic that it detects frequency components that appear repeatedly at same frequencies and does not detect frequency components whose frequencies are constantly changing. Therefore, the frequency components caused by the pumps will not be detected by frequency analysis device 30 if the pumps are operated by fluctuating their rotation frequencies within a certain range. Thus, only the frequency component of the pressure wave caused by the patient's heartbeat will appear in the output of frequency analysis device 30. If the rotation frequency of a pump is fluctuated within a certain range, in particular, with a frequency synchronizing with the sampling frequency of the FFT, the frequency component caused by the pump can be removed more efficiently than when it is not synchronized.

A further embodiment is based on a principle, which is different from those methods above. A sharply changing frequency component can be reasonably assumed to be caused by an anomaly of the blood vessel access, i.e., a disconnection of the vascular cannula, on the ground that neither the frequency components caused by the mechanical devices such as a blood pump nor the frequency component caused by the heartbeat do not change sharply. This method is based on the assumption that the disconnection of a vascular cannula causes an abrupt disappearance of the frequency component caused by the heartbeat. More specifically, it stores the spectrum obtained by the FFT analysis of the frequency components as the first spectrum, and store the spectrum obtained approximately 1 second afterwards by the FFT analysis as the second spectrum.

Compare the first spectrum and the second spectrum, or more specifically, obtain the difference between the frequency components that constitute the first spectrum and the frequency components that constitute the second spectrum by subtracting one from the other. If the vascular cannula is engaged, there will be no substantial differences between the first and second spectrums, so that no frequency component should be remaining after said subtraction. However, if the vascular cannula is disconnected, the frequency component caused by the heartbeat exists in the first spectrum but the same does not exist in the second spectrum, so that the subtraction should produce the frequency component caused by the heartbeat as a remainder.

The reason why the intensity of the frequency component is included as a judgment element here is that the first and second spectrums can never be exactly identical because of noises and the performance limitations of the medical devices. It is useful for the system make a correct judgment by avoiding such a disturbance factor. The method is based on the notion that a change in the intensity of the frequency component of the heartbeat that occurs when the vascular cannula disconnects is much greater than the fluctuations of the frequency components caused by noises and such, which are generally quite small, so that the disconnection of the vascular cannula can be detected securely by monitoring the intensity.

The basic working procedure of the present invention will be described below based on the basic principles described above.

The method includes measuring a spectrum consisting of all the frequency components containing the pressure waves caused by the heartbeat and the blood pump by analyzing the pressure waves being applied on the blood circulating through the extracorporeal circulation circuit with the FFT analysis.

Identifying the frequency component of the pressure wave caused by the mechanical devices that affect the pressure of the circulating fluid such as the blood pump and the dialysate circulation pump other than the heartbeat. There are several methods of identifying the frequency components of the pressure wave that are caused by the mechanical devices.

The spectrum of the frequency components due to the mechanical devices that are identified above are removed from the spectrum comprising a mixture of all kinds of frequency components obtained above, and identifying the resultant frequency component as the frequency component due to the patient's heartbeat.

The intensity of the frequency component caused by the heartbeat obtained above, compared with the standard value preset for identifying an anomaly in the blood vessel access, and deciding that an anomaly exists in the blood vessel access if it is smaller than the standard value.

The above is the basic working procedure of the present invention and a preferable embodiment of the invention will be described in more detail by referring to the accompanying drawings.

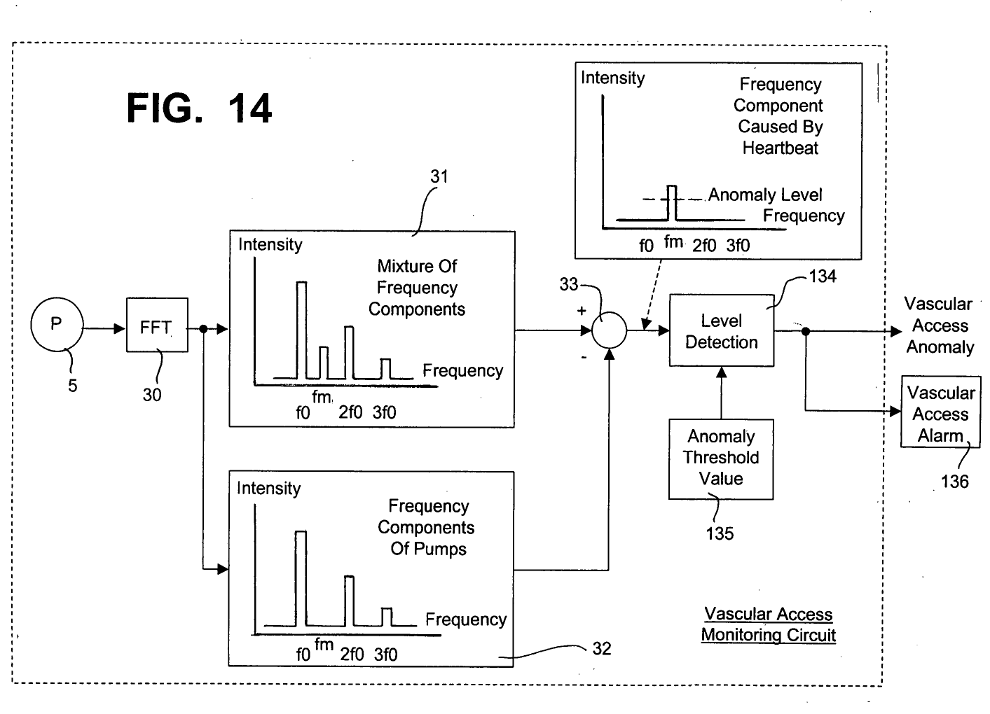

FIG. 14 illustrates a blood vessel access monitoring circuit according to an embodiment of the invention. Although the blood vessel access monitoring circuit can be realized as either a hardware system or a software system, control circuit 11 typically consists of a microcomputer, so that the blood vessel access monitoring circuit can be configured as a software system using said microcomputer, making it unnecessary to add any hardware and lowering the cost of the invention.

FIG. 14 shows frequency analysis devices 30 receiving pressure waveform data of the circulating blood obtained by the pressure detection, devices arterial pressure sensor 5. Frequency analysis devices 30 can be realized either as a software program using a microcomputer, or as a hardware system such as a specially designed IC for FFT analysis. Although the use of an IC may be undesirable from the cost standpoint, it has advantages such that it provides a faster analysis speed and that it creates a smaller burden the microcomputer's CPU. If the FFT analysis is handled by a software program, there is no need for adding hardware device to implement the invention. This can provide for a lower device cost and not alter the external shape of the device.

A certain time period can be set for the FFT analysis, frequency analysis device 30 is provided with a function to set up a certain time period for the FFT analysis. FIG. 2, for example, the FFT is applied for a period of 0 to 5 seconds. The longer the time period, the more repetitions of waveform are entered for the FFT analysis.

The output of frequency analysis 30 is connected to a storage device 31. Storage device 31 stores the spectrum containing a mixture of frequency components caused by the mechanical devices such as blood pump 3 and the dialysate circulation pump and the frequency component caused by the heartbeat obtained by applying the FFT analysis by frequency analysis device 30 to the pressure wave data detected by arterial pressure sensor 5. The contents of storage device 31 are constantly updated with the latest data while the dialysis continues. Since storage device 31 is for temporarily storing the output data of frequency analysis device, storage device 31 can be built into frequency analysis device 30.

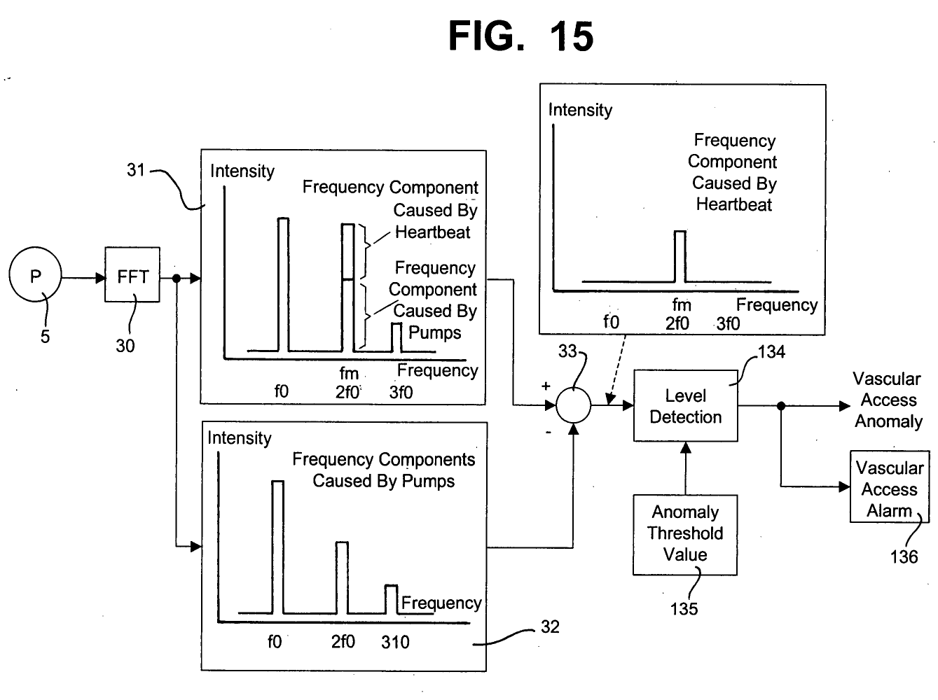

What is important here is that overlapping frequency components caused by the mechanical devices and frequency component caused by the heartbeat are stored in storage device 31 as a spectrum resulting from the addition of the two kinds of frequency components. Therefore, the frequency component caused by the heartbeat can be extracted by removing the frequency components caused by the mechanical devices from the total frequency components. It was not possible to extract only a specified frequency component from a group of overlapping frequency components in the prior art. An embodiment where a plurality of frequency components are overlapping will be described in detail referring to FIG. 15.

On the other hand, the removing consists of a second storage device 32 and a subtraction device 33. Second storage device 32 stores only the spectrum of the frequency components caused by the mechanical devices such as blood pump 3 and the dialysate circulation pump obtained by the FFT analysis using frequency analysis device 30 from the pressure wave data detected by arterial sensor 5 prior to installing arterial cannula 1 and venous cannula 10 to the blood vessel, i.e., prior to installing blood vessel accesses.

Only the spectral data of the frequency components caused by the mechanical devices, which were measured before arterial cannula 1 and venous cannula 10 are attached prior to the start of the dialysis, is stored in second storage device 32, and the same data can be used throughout the dialysis. Once the spectral data of the frequency components due to the mechanical devices are obtained and stored into second storage device 32, as mentioned above, there is no need to obtain the data each time when a dialysis is performed until the mechanical devices of the dialysis device are replaced or deteriorated.

Subtraction device 33, being connected to storage device 31 and second storage device 32, is capable of subtracting the frequency components caused by only the mechanical devices, such as blood pump 3 and the dialysate circulation pump, which are stored in second storage device 32, from the spectrum containing a mixture of the frequency components owing to the mechanical devices and the frequency components owing to the heartbeat, which are stored in storage device 31, to obtain only the frequency components caused by the heartbeat.

The judgment for judging anomaly of the blood vessel access consists of an anomaly judgment setup device 135 and a level detection device 134. Anomaly judgment setup device 135 is preset to a specific intensity of the frequency component caused by the heartbeat, specifically an anomaly judgment value, which is a low value even as the blood pressure of a normal hypotension case, for example, 10 mmHg. When the intensity of the frequency component caused by the heartbeat outputted by subtraction device 33 is lower than the anomaly judgment value indicated by anomaly judgment setup device 135 when the frequency component caused by the heartbeat is compared with the anomaly judgment value at level detection device 134. An anomaly of the blood vessel access, such as the disconnection of the vascular cannula, has occurred. This completes the description of the blood vessel access monitoring circuit. The circuit sends a signal to a blood vessel access alarm 136 thus efficiently causing it to issue an alarm to medical professionals and to such a system as a central monitoring device which centrally controls a plurality of dialysis devices.

The following is a description referring to FIG. 15 for an embodiment where the rotation frequencies caused by blood pump 3 match with the pulse rate so that their frequency components are overlapping each other. Contrary to the prior art, the present embodiment is capable of detecting the presence of anomaly in the blood vessel access based on the frequency component based on the patient's heartbeat by identifying said frequency component without fail even when the patient's pulse rate is very close to or overlapping the rotation frequencies of blood pump 3 or dialysate circulation pump.

FIG. 15 is a diagram showing a blood vessel access monitoring circuit of an embodiment of the present invention described above. The frequency fₘ of the frequency component caused by the patient's heartbeat overlaps a pressure wave with a frequency of twice the frequency f₀ of the frequency component caused by the rotation of blood pump 3.

Second storage device 32 stores only the spectrum of the frequency components of the pressures applied to the blood caused by the mechanical devices such as blood pump 3 and the dialysate circulation pump obtained by the FFT analysis using frequency analysis device 30. The pressure wave data is detected by arterial sensor 5 prior to installing arterial cannula 1 and venous cannula 10 to the blood vessel, i.e., prior to installing blood vessel accesses. This is similar to the embodiment shown in FIG. 14.

In storage device 31, however, the frequency fₘ of the frequency component caused by the patient's heartbeat overlaps with the pressure wave with the frequency of 2f₀, i.e., twice the frequency of the frequency component caused by the rotation of blood pump 3, as shown in FIG. 15. It has hitherto been impossible to identify the pressure wave caused by the heartbeat in such a case, as the device used in the prior art, such as the band-pass filter, removes the pressure wave caused by blood pump 3 having a frequency of 2f₀ together with the pressure wave caused by the heartbeat having a frequency of fₘ.

In contrast, in the present embodiment identifies, only the frequency component caused by the heartbeat as the output of subtraction device 33 as shown in FIG. 15 even when the frequency component caused by the heartbeat overlaps with the frequency components caused by the mechanical devices such as pumps. The frequency components caused by the mechanical devices are removed from the spectrum of storage device 31. The remaining process is to compare the intensity of the frequency component caused by the heart beat with the anomaly judgment value of anomaly judgment setup device 135 in order to judge if there is any anomaly in the blood vessel access.

The present embodiment is effective to monitor the blood vessel access even when the frequency of the pressure wave caused by the heartbeat overlaps the frequency of the pressure wave caused by the mechanical device such as a blood pump, which has been impossible in the prior art. It can also materialize a correct monitoring of the blood vessel access unaffected by temporary pressure wave fluctuations caused by irregular motions that occur in case when the patient turns over in bed.

The above embodiment shows a method of measuring only the frequency components caused by blood pump 3 and the dialysate circulation pump prior to the installation of the blood vessel accesses as a method of obtaining only the frequency components caused by the heartbeat. The method includes subtracting the frequency components caused by blood pump 3 and the dialysate circulation pump from the spectrum containing a mixture of the frequency components caused by the mechanical devices such as blood pump 3 and the dialysate circulation pump and the frequency components caused by the heartbeat.

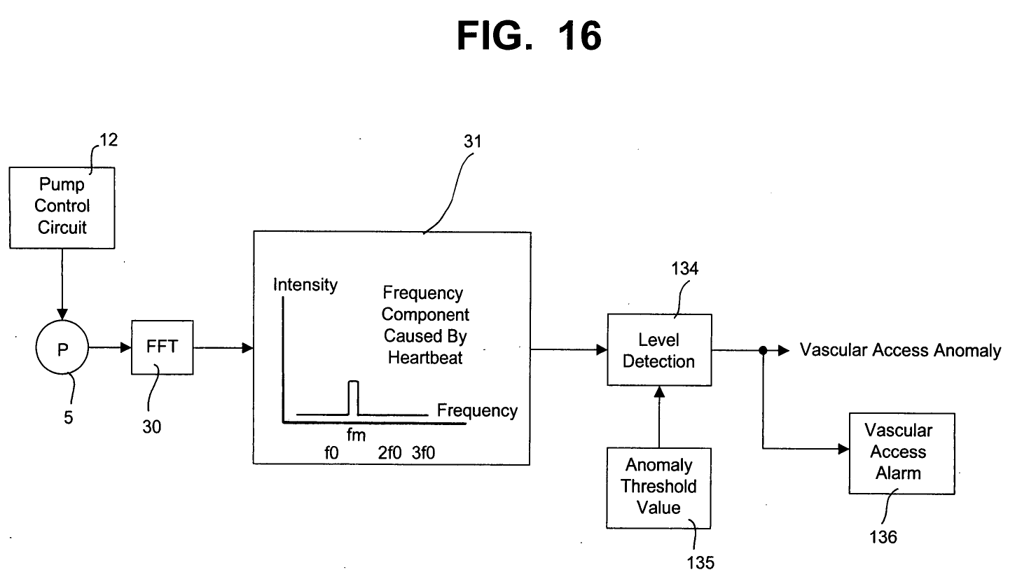

As an embodiment of the above method, there is a method based on control circuit 11 issuing a control instruction to pump control circuit 12 in order to operate blood pump 3 and the dialysate circulation pump by fluctuating their rotational frequencies within a certain range of the standard frequency respectively suited for the patient's dialysis condition that does not burden the patient while executing dialysis with arterial cannula 1 and venous cannula 10 properly installed. The FFT analysis is used here for its characteristic that the FFT detects only those frequency spectrums that appear repeatedly at exact same frequencies and does not detect frequency components whose frequencies are constantly changing. Since pump control circuit 12 causes the pumps to rotate with fluctuating rotation frequencies as described above in this case, only the frequency spectrum caused by the heartbeat appears on storage device 31, as shown in FIG. 16. Thus, only the frequency spectrum caused by the heartbeat can be identified. Therefore, the output of storage device 31 is directly entered into level detection 134.

When the rotation frequencies of blood pump 3 and the dialysate circulation pump are fluctuated within a certain range respectively, in particular, with a frequency synchronizing with the sampling frequency of the FFT, the frequency components caused by said pumps can be removed more efficiently than when they are not synchronized with the sampling frequency.

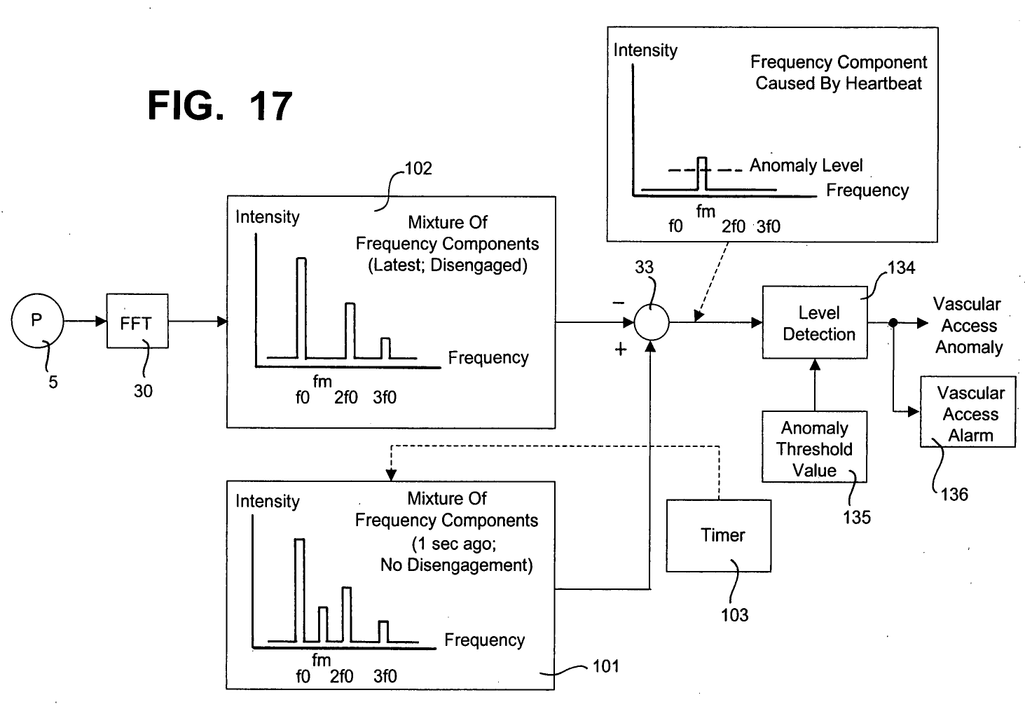

A further removal method will be described below referring to an embodiment shown in FIG. 17. The pressure of the blood to be dialyzed is detected by arterial pressure sensor 5, a pressure detection device, and its pressure data is transmitted to frequency analysis device 30 to be processed by a frequency analysis, i.e., an FFT analysis in this embodiment. The spectrum consisting of frequency components after the FFT analysis is then stored in second memory device 102. The spectrum stored in second memory device 102 is then transmitted to second memory device 101. After a specified time, exactly 1 second after in this embodiment timed by timer 103, the spectrum of 1 second ago stored in memory device 101 is extracted and transmitted to subtraction device 33.

Subtraction device 33 calculates the difference between the latest spectrum of second memory device 102 and the spectrum of 1 second ago stored in second memory device 101. If there is no anomaly, such as disconnection of the vascular cannula or twisting of the blood tube, there is no difference between the frequency components of the latest spectrum stored in memory device 102 and the frequency component of the spectrum of 1 second ago stored in first memory device 101 and no frequency component should exist as the remainder of the subtraction.

However, if there is any anomaly such as disconnection of the vascular cannula or twisting of the blood tube, the frequency component caused by the heartbeat is missing from the latest spectrum stored in second memory device 102, while it exists in the spectrum of 1 second ago stored in first memory device 101, so that the operation by subtraction device 33 should leave the frequency component caused by the heartbeat as the remainder.

However, even if there is no anomaly, there can be a minute frequency component for each frequency due to noise between the latest spectrum stored in second memory device 102 and the spectrum of 1 second ago stored in first memory device 101 as mentioned above. In order to prevent the system from making a misjudgment of such a minute residual frequency component as a sign of a disconnection of the vascular cannula, level detection device 134 compares the intensity of each residual frequency component with the anomaly threshold value indicated by anomaly judgment setup device 135, once completed, it determines that an anomaly such as a disconnection of the vascular cannula exists in the blood vessel access even a single frequency component is found to be greater than the anomaly threshold value.

Although the above description detects anomalies of the arterial access by detecting the circulating blood to be measured with arterial pressure sensor 5, anomalies of the venous access can also be monitored from the spectrum diagram shown in FIG. 3(B) using the embodiments shown in FIG. 14, FIG. 16, and FIG. 17.

Anomalies of arterial blood vessel access such as a disconnection of the arterial cannula can be detected by the arterial pressure data obtained by the arterial pressure sensor, while anomalies of venous blood vessel access such as a disconnection of the venous cannula can be detected by the venous pressure data obtained by the venous pressure sensor. Therefore, the invention has an advantage that it does not require both the arterial pressure data and the venous pressure data in order to detect anomalies of the blood vessel access, but rather, as long as a pressure sensor exists on either one of the arterial and venous sides, it can monitor the blood vessel access on the side the sensor exists. This is an excellent advantage of the invention because a pressure sensor exists only on one side in some medical devices.

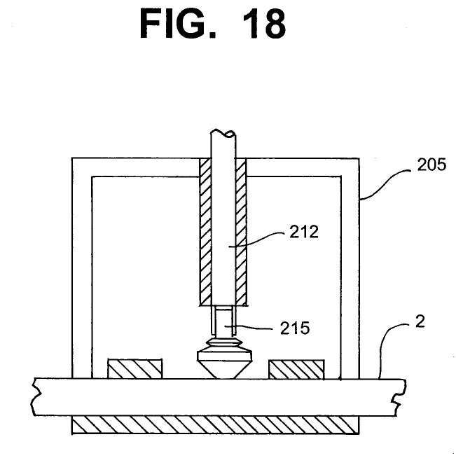

Moreover, although the above description assumed that a pressure sensor is attached to a drip chamber as a means of detecting the pressure of the fluid being transported or the circulating blood in case of a dialysis device, the invention is not limited to such a sensor as long as the pressure of the fluid being transported can be measured. For example, the invention is applicable no matter where the pressure of the blood circulating through the dialysis device is measured. For example, the blood tube expands or contracts depending on the pressure of the blood as the blood moves through the blood tube. Therefore, it is possible to perform the same function as measuring the pressure of the blood being transferred by measuring the expansion and contraction of the blood tube. A specific sensor for measuring the expansion and contraction of blood tube 2 is shown in FIG. 18. A tube deformation measurement sensor 205 transmits the deformation of the blood tube that expands or contracts depending on the pressure change in the blood being transported to a variable rod 215 and its displacement is detected by a displacement sensor 212. The detail of this device is disclosed by JP'590 and JP'665.

It is also possible to monitor the blood vessel access by measuring the pressure of the dialysate and specifying the frequency component caused by the heartbeat contained in the pressure wave instead of directly measuring the pressure of the blood because the pressure of the circulating blood is transmitted to the dialysate via the dialyzer in the dialysis device.

Also, the present invention can be used for checking whether the vascular cannula is securely attached before starting the dialysis in addition to monitoring anomalies in the blood vessel access during the dialysis. This procedure can be used to measure the frequency components caused by the patient's heartbeat while the vascular cannula is connected to the patient and the mechanical devices such as pumps are not operating, so that it is possible to judge that the blood vessel access is working normal by confirming the frequency components caused by the patient's heartbeat. It also provides a benefit of assuring the safe operation of the dialysis device by using the judgment for the interlock of the operation of the dialysis device.

As described above, the present embodiment can be used to monitor the patient's blood vessel access in the dialysis device during a dialysis operation, detect a major accident of the patient such as a disconnection of the vascular cannula as soon as it occurs, and quickly take a necessary measures to prevent any catastrophic circumstances. The invention also provides a benefit of detecting accidents that can degrade the dialysis operation such as twisting of the blood tube in addition to detection of major accidents such as a disconnection of the vascular cannula.

The reason that the present embodiment is superior to the similar methods of the prior art of monitoring the blood vessel access that detect pressure waves caused by the heartbeat for the dialysis device is that it can monitor the blood vessel access without fail even when the pulse rate overlaps the rotation frequencies of blood pumps, etc. Also, the use of the FFT analysis in this embodiment provides an excellent feature that the patient's blood vessel access can be measured more securely as it prevents the analyzer's malfunction which might otherwise be caused by pressure waves applied to the blood as a result of irregular motions of the patient, as in the case of the patient's turnover in the bed.

Moreover, although it has been described so far about a case wherein the FFT analysis is used as the frequency analysis device for separating the frequency component of the pressure wave caused by the heartbeat from the frequency components of the pressure wave caused by the mechanical devices such as the pumps, other frequency analysis device that are capable of differentiating the frequencies of these pressure waves other than the FFT analysis. For example, the normal Fourier analysis and the MEM (maximum entropy method), can be used for the same purpose.

The present invention can be applied not only for monitoring the blood vessel access of the dialysis device, it can also be applied to various other medical devices such as the infusion pump device and the artificial heart-lung machine.

FIG. 12 shows an embodiment applied to a fluid infusion device. A fluid infusion device is different from a dialysis device in that it is neither for circulating blood nor for measuring the blood pressure of circulating blood. In case of a fluid infusion device, a solution infused into a blood vessel corresponds to the fluid to be transported, the pressure applied to the infusion fluid is detected by pressure detecting device 5, and a mixture of frequency component of the pressure wave caused by infusion pump 300 and a frequency component of the pressure wave caused by the heartbeat exists. The invention extracts only the frequency component of the pressure wave caused by the heartbeat from the mixture in order to detect the disconnection of vascular cannula.

Thus, the use of this invention can be used for dialysis devices, artificial heart-lung machines, infusion devices, or blood transfusion devices providing a device of monitoring anomalies of blood vessel accesses.

As described above, the blood vessel access monitoring method in medical devices and the medical device using the method according to the present invention make it possible to monitor the condition of the blood vessel access in the medical device accurately without requiring special equipment and without causing extra burdens to the patient or medical professionals to secure the safety of the patient by monitoring the intensity of the frequency spectrum of the pressure wave caused by the patient's heartbeat from the pressured applied to the fluid of the medical device.

### INDUSTRIAL APPLICATION

The present invention provides a method of accurately and continuously measuring the pulse rate and blood pressure of a patient being treated with a medical device, which is connected to the patient's blood vessel through a blood vessel access and has a mechanical device for applying a pressure to transport a fluid to said blood vessel, without burdening the patient or medical professionals and without requiring any additional equipment by means of identifying using the frequency analysis the frequency component of the pressure wave caused by the patient's heartbeat in the fluid's pressure wave, as well as a medical device using said method.

Moreover, the blood vessel access monitoring method in medical devices and the medical device using said method according to the present invention make it possible to monitor the condition of the blood vessel access in the medical device accurately without requiring special equipment and without causing extra burdens to the patient or medical professionals to secure the safety of the patient by monitoring the intensity of the frequency spectrum of the pressure wave caused by the patient's heartbeat from the pressured applied to the fluid of the medical device.

Hence obvious changes may be made in the specific emobodiment of the invention described herein, such modification being within the spirit and scope of the invention claimed, it is indicated that all matter contrained herein as an illustrated and not as limiting in scope.

1. A method of measuring a patient's pulse rate in a medical device, which is connected the patient's blood vessel by a blood vessel access and has a mechanical device for applying a pressure to a fluid in order to transport the fluid to said blood vessel, comprising steps of:
measuring the pressure of said fluid;
detecting a spectrum consisting of frequency components comprising the step of applying a frequency analysis to said measured pressure for a period of time;
identifying a frequency component caused by the patient's heartbeat by removing a frequency component caused by said mechanical device from said spectrum; and
measuring the patient's pulse rate from said frequency component caused by the patient's heartbeat.

2. A method of measuring a patient's pulse rate according to claim 1, comprising steps of:
measuring the pressure of said fluid prior to installing the blood vessel access;
detecting the frequency component caused by said mechanical device comprising the step of applying a frequency analysis to said measured pressure for a certain period of time; and
removing the frequency component caused by said mechanical device from said spectrum.

3. A method of measuring a patient's pulse rate according to claim 1, comprising steps of:
transmitting an operation frequency of said mechanical device to identify the frequency caused by said mechanical device; and
removing the operation frequency component caused by said mechanical device from said spectrum.

4. A method of measuring a patient's pulse rate in a medical device, which is connected the patient's blood vessel by a blood vessel access and has a mechanical device for applying a pressure to a fluid in order to transport the fluid to said blood vessel, comprising steps of:
measuring the pressure of said fluid prior to installing the blood vessel access while causing a pump to fluctuate a rotating frequency within a certain range of a standard frequency;
detecting a spectrum consisting of a frequency component caused by the patient's heartbeat by applying a frequency analysis to said measured pressure for a period of time; and
measuring the patient's pulse rate from said frequency component caused by the patient's heartbeat.

5. A method of measuring blood pressure in a medical device, which is connected the patient's blood vessel by a blood vessel access and has a mechanical device for applying a pressure to a fluid in order to transport the fluid to said blood vessel, comprising steps of:
measuring the pressure of said fluid;
detecting a spectrum consisting of frequency components by applying a frequency analysis to said measured pressure for a period of time;
identifying a frequency component caused by the patient's heartbeat by removing a frequency component caused by said mechanical device from said spectrum; and
measuring the patient's blood pressure from an intensity of said frequency component caused by the patient's heartbeat.

6. A method of measuring a patient's blood pressure according to claim 5, comprising steps of:
measuring the pressure of said fluid prior to installing the blood vessel access;
detecting the frequency component caused by said mechanical device by applying a frequency analysis to said measured pressure for a period of time; and
removing the operation frequency component caused by said mechanical device from said spectrum.

7. A method of measuring a patient's blood pressure according to claim 5, comprising steps of:
transmitting an operation frequency of said mechanical device to identify the frequency caused by said mechanical device; and
removing the operation frequency component caused by said mechanical device from said spectrum.

8. A method of measuring a patient's blood pressure in a medical device, which is connected the patient's blood vessel by a blood vessel access and has a mechanical device for applying a pressure to a fluid in order to transport the fluid to said blood vessel, comprising steps of:
measuring the pressure of said fluid prior to installing the blood vessel access while causing a pump to fluctuate a rotating frequency within a certain range of a standard frequency;
detecting a spectrum consisting of a frequency component caused by the patient's heartbeat by applying a frequency analysis device to said measured pressure for a period of time; and
measuring the patient's blood pressure from an intensity of said frequency component caused by the patient's heartbeat.

9. A medical device, which is connected the patient's blood vessel by a blood vessel access and has a mechanical device for applying a pressure to a fluid in order to transport the fluid to said blood vessel, characterized in having a pulse rate measuring circuit comprising:
a pressure detection device measuring the pressure of said fluid;
a frequency analysis device detecting a spectrum comprising frequency components by applying a frequency analysis device to said measured pressure for a period of time;
a removal device removing a frequency component caused by the patient's heartbeat by removing a frequency component caused by said mechanical device from said spectrum; and
a pulse rate conversion device converting the frequency of said frequency component caused by the patient's heartbeat into the patient's pulse rate.

10. A medical device according to claim 9, wherein a plurality of said periods of time can be preset, and further comprising:
a pulse rate measuring circuit for measuring a pulse rate at each preset time period.

11. A medical device according to claim 10 further comprising:
a pulse rate warning circuit for issuing an alarm when a pulse rate measured by said pulse rate measuring circuit falls outside of a predetermined pulse rate normal value range.

12. A medical device, connected to the patient's blood vessel by a blood vessel access and has a mechanical device for applying a pressure to a fluid in order to transport the fluid to said blood vessel, characterized in having a blood pressure measuring circuit comprising:
a pressure detection device measuring the pressure of said fluid,
a frequency analysis device detecting a spectrum consisting of frequency components by applying a frequency analysis to said measured pressure for a period of time;
a removal device removing a frequency component caused by the patient's heartbeat by removing a frequency component caused by said mechanical device from said spectrum, and
a blood pressure conversion device converting an intensity of said frequency component caused by the patient's heartbeat into the patient's blood pressure.

13. A medical device according to claim 12, wherein a plurality of said periods of time can be preset, and further comprising:
a blood pressure measuring circuit for measuring a blood pressure at each preset time period.

14. A medical device according to claim 13 further comprising:
a blood pressure warning circuit for issuing an alarm when a blood pressure value measured by said blood pressure measuring circuit falls outside of a predetermined blood pressure normal value range.

15. A medical device according to claim 9, wherein the pressure of said fluid is measured on at least one of an arterial side fluid and a venous side fluid.

16. A medical device according to claim 12, wherein the pressure of said fluid is measured on at least one of an arterial side fluid and a venous side fluid.

17. A method of monitoring a blood vessel access in a medical device, which is connected the patient's blood vessel by a blood vessel access and has a mechanical device for applying a pressure to a fluid in order to transport the fluid to said blood vessel, comprising steps of:
measuring the pressure of said fluid;
detecting a spectrum consisting of frequency components by applying a frequency analysis device to said measured pressure for a period of time;
identifying a frequency component caused by the patient's heartbeat by removing a frequency component caused by said mechanical device from said spectrum, and
monitoring anomalies of said blood vessel access by judging an intensity level of said frequency component caused by the patient's heart beat.

18. A method of monitoring a blood vessel access according to claim 17, wherein said removal device:
measures said pressure of the fluid prior to installation of said blood vessel access;
detects said frequency component caused by said mechanical device by applying a frequency analysis to said measured pressure for a certain period of time; and
removes said frequency component caused by said mechanical device from said spectrum.

19. A method of monitoring a blood vessel access in a medical device, which is connected the patient's blood vessel by the blood vessel access and has a mechanical device for applying a pressure to a fluid in order to transport the fluid to said blood vessel, comprising steps of:
measuring the pressure of said fluid prior to installing the blood vessel access while causing a pump to fluctuate a rotating frequency within a certain range of a standard frequency;
detecting a spectrum consisting of a frequency component caused by the patient's heartbeat by applying a frequency analysis device to said measured pressure for a period of time; and
monitoring anomalies of the blood vessel access comprising the step of judging an intensity level of said frequency component caused by the patient's heart beat.

20. A method of monitoring a blood vessel access in a medical device, which is connected the patient's blood vessel by a blood vessel access and has a mechanical device for applying a pressure to a fluid in order to transport the fluid to said blood vessel, comprising steps of:
measuring the pressure of said fluid;
detecting a first spectrum consisting of a frequency component by applying a frequency analysis device to said measured pressure for a first period of time;
storing said first spectrum;
detecting a second spectrum consisting of a frequency component by applying a frequency analysis to said measure pressure for a second period of time after said first period of time;
storing said second spectrum;
obtaining a difference between the frequency component of said first spectrum and the frequency component of said second spectrum; and
judging the intensity level of said difference.

21. A method of monitoring a blood vessel access according to claim 17, wherein the pressure of said fluid is measured on at least one of an arterial side fluid and a venous side fluid.

22. A method of monitoring a blood vessel access according to claim 18, wherein the pressure of said fluid is measured on at least one of an arterial side fluid and a venous side fluid.

23. A method of monitoring a blood vessel access according to claim 19, wherein the pressure of said fluid is measured on at least one of an arterial side fluid and a venous side fluid.

24. A method of monitoring a blood vessel access according to claim 20, wherein the pressure of said fluid is measured on at least one of an arterial side fluid and a venous side fluid.

25. A medical device, connected a patient's blood vessel by a blood vessel access and has a mechanical device for applying a pressure to a fluid in order to transport the fluid to said blood vessel, having a blood vessel access monitoring circuit comprising:
a pressure detection device measuring the pressure of said fluid,
a frequency analysis device detecting a spectrum consisting of each frequency component by applying a frequency analysis device to said measured pressure for a period of time;
a removal device removing a frequency component caused by the patient's heartbeat by removing a frequency component caused by said mechanical device from said spectrum, and
a judgment device judging anomalies of said blood vessel access by measuring an intensity level of said frequency component caused by the patient's heartbeat.

26. A medical device according to claim 25, wherein said removal device:
measures said pressure of the prior to installation of said blood vessel access,
detects the frequency component caused by said mechanical device by applying a frequency analysis to the measured pressure for a certain period of time; and
removing said frequency component caused by said mechanical device from said spectrum.

27. A medical device connected a patient's blood vessel by a blood vessel access and has a mechanical device for applying a pressure to a fluid in order to transport the fluid to said blood vessel, having a blood vessel access monitoring circuit comprising:
a pressure detection device measuring pressure of said fluid;
a frequency analysis device detecting a first spectrum consisting of frequency components by applying a frequency analysis device to said measured pressure for a first period of time;
a first storage device for storing said first spectrum;
said frequency analysis device detecting a second spectrum consisting of frequency components by applying a frequency analysis device to said measured pressure for a second period of time;
a second storage device for storing said second spectrum; and
a judgment device judging the intensity level of the difference between said frequency component of said first spectrum and said frequency component of said second spectrum.

28. A medical device according to claim 25 further comprising:
a blood vessel access warning circuit for issuing an alarm when said judgment device detects an anomaly.

29. A medical device according to claim 25, wherein the pressure of said fluid is measured on at least one of an arterial side fluid and a venous side fluid.

30. A medical device according to claim 27 further comprising:
a blood vessel access warning circuit for issuing an alarm when said judgment device detects an anomaly.

31. A medical device according to claim 27, wherein the pressure of said fluid is measured on at least one of an arterial side fluid and a venous side fluid.
